(19) United States
(12) Patent Application Publication
(10) Pub. No.: US 2014/0057837 A1
(43) Pub. Date: Feb. 27, 2014

(54) METHODS AND COMPOSITIONS FOR THE "BROWNING" OF WHITE FAT

(71) Applicants: Matthew During, Columbus, OH (US); Lei Cao, Columbus, OH (US)

(72) Inventors: Matthew During, Columbus, OH (US); Lei Cao, Columbus, OH (US)

(21) Appl. No.: 13/971,968

(22) Filed: Aug. 21, 2013

Related U.S. Application Data
(60) Provisional application No. 61/691,558, filed on Aug. 21, 2012.

(51) Int. Cl. A61K 38/18; A61K 31/353; A61K 31/24; A61K 31/135; A61K 31/138; A61K 31/7088; A61K 31/4045

(52) U.S. Cl. CPC A61K 38/185; A61K 31/7088; A61K 31/353; A61K 31/4045; A61K 31/135; A61K 31/138; A61K 31/24
USPC 514/4.8; 514/44 R; 514/456; 514/419; 514/654; 514/653; 514/510

(57) ABSTRACT

Methods for increasing the amount of brown adipose tissue in a subject, of increasing the ratio of brown fat to white fat in a subject, or for effecting a change in white adipose tissue to become brown adipose tissue in a subject include the administration of one or more agents. The agent can increase or induce hypothalamic expression of BDNF, can be a TrkB receptor agonist or a beta-3 adrenergic receptor agonist, or encode an agonist that modulates a hypothalamic-adipocyte axis.

### CROSS-REFERENCE TO RELATED APPLICATIONS

This application claims the benefit of priority to U.S. Provisional Application No. 61/691,558 filed Aug. 21, 2012, the entire content of which is herein incorporated by reference in its entirety.

### STATEMENT REGARDING FEDERALLY SPONSORED RESEARCH

This invention was made with government support under Grant No. RO1 NS 44576 awarded by National Institute of Neurological Diseases and Stroke. The government has certain rights in the invention.

### BACKGROUND OF THE INVENTION

Obesity and metabolic syndrome are rapidly becoming major global health, social, and economic problems with substantial morbidity and mortality (Batsis et al., 2007). Obesity results from chronic excess energy intake over energy expenditure and is controlled by variable and complicated interactions between genetic background, environmental factors, behavioral factors, and socioeconomic status.

Two types of adipose tissue have been found in mammals, white adipose tissue (WAT) and brown adipose tissue (BAT). White adipose tissue and brown adipose tissue are different at functional, morphological, and molecular levels. Brown adipose tissue dissipates energy directly as heat through uncoupling fatty acid oxidation from ATP production by uncoupling protein-1 (UCP-1), regulates body temperature, and is also involved in the control of body weight. White adipose tissue accumulates excess energy as triacylglycerols and was previously viewed as a passive organ with relatively simple functions such as energy storage, heat insulation, and mechanical cushioning. However, white adipose tissue may be a versatile and more complex organ with many functions other than energy balance and may be highly adaptive to external stimuli.

Additionally, brown adipocyte-like cells are found in white adipose tissue of rodents and humans. These cells with a multilocular morphology and expressing the brown adipocyte-specific UCP-1 are called brown-in-white (brite) cells, beige cells, or adaptive or recruitable brown adipocytes. Although the precise origin of these cells is not fully defined, the development of these thermogenic-competent cells in white adipose tissue is greatly enhanced in response to chronic cold exposure or prolonged β-adrenergic stimulation, and the occurrence of these cells is associated with resistance to obesity and metabolic diseases.

### SUMMARY OF THE INVENTION

In one embodiment, a method for increasing the amount of brown adipose tissue in a subject having a low amount of brown adipose tissue includes the step of administering a TrkB receptor agonist or a nucleotide sequence encoding a TrkB receptor agonist to the subject having a low amount of brown adipose tissue. The TrkB receptor agonist or expression of the TrkB receptor agonist increases the amount of brown adipose tissue in the subject.

The TrkB receptor agonist can be a peptide such as BDNF, Neurotrophin-4, and Neurotrophin-3, or can be 7,8-dihydroxyflavone, N-acetylserotonin, Amitriptyline, 4′-Dimethylamino-7,8-dihydroxyflavone, LM22A-1, LM22A-2, LM22A-3, and LM22A-4. The TrkB receptor agonist can be administered systemically, centrally, directly into a brain, or a combination thereof in an amount sufficient to activate the TrkB receptor. The TrkB receptor agonist or expression of the TrkB receptor agonist modulates sympathetic nervous system activity, such as a hypothalamic BDNF signaling pathway, thereby increasing the amount of brown adipose tissue in the subject.

In one aspect, a method for increasing the amount of brown adipose tissue in a subject includes the step of administering a beta-3 adrenergic receptor agonist or a nucleotide sequence encoding a beta-3 adrenergic receptor agonist to a subject having a low amount of brown adipose tissue.

In one embodiment, the subject may be suffering from diet-induced obesity, and shows enhanced weight loss, a prevention in weight gain, an increased basal or resting metabolic rate, or combination thereof.

In one aspect, the beta-3 adrenergic receptor agonist is one or more of Octopamine, Amibegron, Solabegron, Nebivolol, L796568, CL-316243, LY-368842, and Ro40-2148. The beta-3 adrenergic receptor agonist can be administered orally or by injection in an amount sufficient to activate the beta-3 adrenergic receptor and can increase the amount of brown adipose tissue in the subject. The beta-3 adrenergic receptor agonist can be administered in an amount sufficient to reduce the amount of white adipose tissue, and can modulate β-adrenergic signaling, thereby causing a browning of white adipose tissue.

In one aspect, a method for increasing the amount of brown adipose tissue in a subject includes the step of administering an agonist or a nucleotide sequence that encodes an agonist that modulates a hypothalamic-adipocyte axis in a subject having a low ratio of brown adipose tissue to white adipose tissue, wherein modulation of the hypothalamic-adipocyte axis increases the amount of brown adipose tissue in the subject. The agonist can be a TrkB receptor agonist or a beta-3 adrenergic receptor agonist.

In one embodiment, a method of preventing weight gain or effecting weight loss in a mammal includes the step of providing an enriched environment to a mammal such that the presence of the enriched environment increases the amount of brown adipose tissue in the mammal.

Other aspects of the invention will become apparent by consideration of the detailed description and accompanying drawings.

### BRIEF DESCRIPTION OF THE DRAWINGS

FIG. 1. EE decreases adiposity of mice fed on NCD. (A) Four weeks EE or wheel running decreased body weight and fat pad mass (n=10 per group). (B) MRI analysis of abdominal fat and lean mass (n=5 per group). Intra-ab., intra-abdominal; Subcu., subcutaneous. (C) EE increased food intake (n=10 per group). (D) EE increased basal resting oxygen consumption (n=8 per group) * P<0.05. (E) EE increased oxygen consumption in retroperitoneal white adipose tissue ex vivo (P<0.05 EE compared to control and running, n=3 per group). Running increased oxygen consumption in brown adipose tissue (P<0.05 running compared to control and EE, n=3 per group). NRF, normalized relative fluorescence. (F) EE and EE no wheel did not decrease body weight whereas wheel running decreased body weight (n=10˜20 per group) * P<0.05 for Running (G) Fat pad mass calibrated to body weight (n=10˜19 per group). (H) Gene expression profile of the PVH after 10 weeks respective housing (n=5 per group). Bars not connected by same letter are significantly different. Data are means±SEM.

FIG. 2. EE induces brown fat molecular features in white fat. Gene expression profile of retroperitoneal white adipose tissue (A) and brown adipose tissue (B) after 4 weeks respective housing (n=5 per group). (C) H&E staining and UCP1 immunohistochemistry of retroperitoneal white adipose tissue. Scale bar: 20 μm. (D) Western blot of retroperitoneal white adipose tissue. (E) Mitochondrial DNA content of retroperitoneal white adipose tissue (n=4 per group) * P<0.05. (F) Gene expression profile of retroperitoneal white adipose tissue after 9 weeks respective housing (n=4 per group). Bars not connected by same letter are significantly different. Data are means±SEM.

FIG. 3. EE prevents DIO. (A-C) EE mice remained lean compared to control mice when fed on HFD. Scale bar: 1 cm. EE mice had less weight gain (D) and fat mass (E) (n=10 per group). (F) Food intake. (G) Biomarkers in serum. (H) Gene expression profile in retroperitoneal white adipose tissue (n=5 per group). P values of significance were shown above the bars. (I-J) Pparg1a and Prdm16 mRNA levels were inversely correlated to retroperitoneal white adipose tissue weight. Filled circles: individual EE mouse, blank circles: individual control mouse. * P<0.05, ** P<0.01, *** P<0.001. Data are means±SEM.

FIG. 4. Long-term EE leads to stronger white fat “browning”. (A) Representatives of brown adipose tissue, epididymal white adipose tissue and retroperitoneal white adipose tissue of control or EE mice after 3 months EE. Scale bar: 1 mm. (B) H&E staining of epididymal white adipose tissue. Scale bar: 20 μm. (C) Gene expression profile of epididymal white adipose tissue (n=4 per group). (D) Immunohistochemical staining of UCP 1 in retroperitoneal white adipose tissue. Scale bar: 200 μm in the upper panels, 20 μm in the lower panels. (E) Gene expression profile of retroperitoneal white adipose tissue (n=4 per group). P values are shown above the bars. (F) Gene expression profile of retroperitoneal white adipose tissue 4 hours after NE injection (n=4 per group). Bars not connected by same letter are significantly different. Data are means±SEM.

FIG. 5. Hypothalamic BDNF mediates EE-induced white adipose tissue to brown adipose tissue transformation. (A) rAAV-mediated gene delivery of BDNF to hypothalamus reproduced EE-associated reduction of adiposity. (B) Representative images of adipose tissues by MRI. (C) MRI analysis of abdominal fat and lean mass (n=4 per group) *P<0.05. Intra-ab., intra-abdominal; Subcu., subcutaneous. (D) Gene expression profile of retroperitoneal white adipose tissue of BDNF-overexpressing mice and YFP control mice. n=4 BDNF, n=S YFP. P values are shown above the bars. (E) Western blot of retroperitoneal white adipose tissue. Data are means±SEM.

FIG. 6. BDNF inhibition blocks the EE-induced white adipose tissue to brown adipose tissue transformation. (A) Increase of adiposity occurred before weight increase in BDNF+/− mice compared to wild-type littermates. n=4 per group. ** P<0.01, +P<0.06. (B) retroperitoneal white adipose tissue gene expression profile of BDNF+/− mice compared to wild type litter mates (n=4 per group). P values are shown above the bars. (C) TrkB.T1-expressing mice were equally obese as DIO mice expressing YFP (n=5 per group). (D) retroperitoneal white adipose tissue gene expression profile of TrkB.T1-expressing mice compared to lean YFP control mice fed with NCD and obese YFP mice fed with HFD (n=5 per group). (E) microRNA targeting BDNF blocked EE-associated molecular features of retroperitoneal white adipose tissue (n=4 per group. Con: control housing, Enr: EE housing). Bars not connected by same letter are significantly different. Data are means±SEM.

FIG. 7. Mechanism of EE-induced white fat “browning”. Diagram depicts mechanisms through which an enriched environment induces the browning of white fat. See Discussion for details.

FIG. 8. (A) Oxygen consumption and physical activity measured by CLAMS (Comprehensive Lab Animal Monitoring System) for 3 days at 22° C. No significant difference in oxygen consumption. P<0.05 for physical activity (Repeated measures ANOVA). Data points are the mean value for eight mice for each interval of measurement. (B) Gene expression profile for the whole hypothalamus dissection after 4 weeks respective housing (n=5 per group). P values with significant or strong trend compared to control mice are shown above the bars.

FIG. 9. Additional gene expression profiles of retroperitoneal white adipose tissue (A) and WAT (B) after 4 weeks enrichment. N=5 per group, * P<0.05. (C) TUNEL assay of retroperitoneal white adipose tissue. Representative images of retroperitoneal white adipose tissue from control or enriched mice with intestine as positive control (apoptotic cells shown in green). Scale bar: 50 μm. (D) Gene expression profiles of epididymal white adipose tissue (D) and inguinal white adipose tissue (E) after 4 weeks respective housing. n=5 per group, * P<0.05.

FIG. 10. Enrichment leads to efficient cold response and 13 blockade abolishes the enrichment-induced molecular signature. (A) Rectal temperature after acute cold exposure. (B) mRNA expression levels of Dio1 and Ppargc1a in brown adipose tissue and retroperitoneal white adipose tissue after cold exposure. n=4 per group. Bars not connected by same letter are significantly different. (C) Propranolol blocked enrichment-associated molecular features of white adipose tissue (n=5 per group). (D) Blood pressure and heart rate measured using a computerized noninvasive tail-cuff manometry system after 4 weeks control or enriched housing. n=5 per group, P=0.192 for systolic BP, P=0.141 for heart rate, repeated measures ANOVA.

FIG. 11. (A) rAAV-mediated gene delivery of BDNF to hypothalamus reproduced enrichment-associated weight change and fat depletion. Scale bar: 1 cm, (B) UCP1 immunohistochemistry of retroperitoneal white adipose tissue of BDNF overexpressing DIO mice. Scale bar: 20 μm. (C) ex vivo oxygen consumption of dissected brown adipose tissue, inguinal white adipose tissue, epididymal white adipose tissue and retroperitoneal white adipose tissue (triplicate each fat pad, n=3 per group, P<0.05). (D) Hypothalamic gene expression profile of BDNF overexpressing mice. n=5 per group. P values with significant or strong trend compared to YFP mice are shown above the bars. (E) Combination of propranolol and SR59230A attenuated AAV-BDNF-induced molecular features of white adipose tissue (n=5 per group). Bars not connected by same letter are significantly different. Veh: vehicle, pro: propranolol, SR: SR59230A.

FIG. 12. (A) Muscle β-adrenergic receptor expressions were not influenced by lower level of BDNF in BDNF+/− mice compared to wild type litter mates. n=4 per group. (B) Enrichment had no significant effect on muscle β-adrenergic receptor expressions. n=5 per group. (C) AAV=TrkB.T1 mice consumed more food. n=10 per group. * P<0.05. (D) Body weight of AAV-TrkB.T1 or AAV-YFP injected mice. n=10 per group. * P<0.05. (E) Hypothalamic gene expression profile of AAV-TrkB.T1 or AAV-YFP injected mice. n=5 per group. P values with significant or strong trend compared to YFP mice are shown above the bars.

### DETAILED DESCRIPTION

It is to be understood that both the foregoing general description and the following detailed description are exemplary and explanatory only and are not restrictive of the invention, as claimed.

In this application, the use of the singular includes the plural unless specifically stated otherwise. In this application, the word “a” or “an” means “at least one” unless specifically stated otherwise. In this application, the use of “or” means “and/or” unless stated otherwise. Furthermore, the use of the term “including,” as well as other forms, such as “includes” and “included,” is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including but not limited to patents, patent applications, articles, books, and treatises are hereby expressly incorporated by reference in their entirety for any purpose. In the event that one or more of the incorporated literature and similar materials defines a term that contradicts that term's definition in this application, this application controls.

As described herein, the present inventors have identified the hypothalamic-adipocyte axis, a nerve and biochemical pathway that begins in the hypothalamus, and ends in white fat cells, as a therapeutic target for obesity that places patients at higher risk for the development of insulin resistance, diabetes, cardiovascular disease including hypertension and ischemic heart disease, osteoarthritis, and cancer. Described are methods for enhancing weight loss or preventing weight gain in a subject by modulating the hypothalamic-adipocyte axis. For example, a subject may be provided with an environment enriched with physical, mental or social opportunities, or may be administered with one or more agonists or nucleotide sequences encoding an agonist of the hypothalamic-adipocyte axis, such as one or more TrkB receptor agonists, nucleotide sequences encoding a TrkB receptor agonist, and beta-3 adrenergic receptor agonists, or any combination thereof.

In some embodiments, environmental enrichment (EE) including, without limitation, physical, mental and social stimulation induces in subjects a molecular and functional switch from white adipose tissue or white fat to brown adipose tissue or brown fat, and weight loss or a reduction or prevention in weight gain. The switch occurs even in the absence of chronic cold or prolonged pharmacological 3-adrenergic stimulation. The molecular characteristics of EE-induced white adipose tissue “browning” include, without limitation, the induction of brown fat molecular switch Prdm16 and brown fat markers such as Ucp1, Ppragc1a, Elovl3, and Cidea; suppression of white fat-enriched gene Resn; with no changes in adipocyte markers shared by both white and brown fat such as Pparg and Ap2.

The development of obesity and weight gain involves adaptive thermogenesis, the regulated production of heat in response to environmental temperature or diet. The inventors discovered a new type of adaptive thermogenesis in response to an enriched environment, which also requires sympathetic nervous system (SNS) activation. Administration of the β-blocker propranolol sufficiently blocks the EE-induced program in white adipose tissue. Although systemic β-blockade affects various tissues, the efficient blockade of molecular signatures in white adipose tissue suggests intact β-adrenergic signaling is required for the EE-induced browning. The inventors discovered a selective SNS regulation of white adipose tissue by EE with no significant effects on blood pressure or heart rate and that SNS outflow to white adipose tissue is increased by hypothalamic BDNF expression signals.

The adipose organ displays considerable plasticity. In basal conditions, the adipose organ uses white and brown adipose tissues to meet the two physiological requirements of heat production and energy storage by brown and white adipose tissues, respectively. In the case of chronic energy surplus, brown adipose tissue or brown fat is able to transform to white adipose tissue or white fat to store more energy molecules, whereas, in the event of chronic cold exposure, white fat can convert to brown fat. In most adult mammals, white fat is prevalent, but the ratio of white fat to brown fat varies with genetic background, sex, age, nutritional status, and environmental conditions. Adult humans have a small amount of brown fat, about 0.05 percent to about 1.0 percent of body weight. However, it has been estimated that 40 to 50 ng of maximally stimulated brown fat in a human could correspond to as much as 20 percent of energy expenditure, equivalent to 20 kg of body weight, over a year. Accordingly, even a small increase in an amount of brown fat could have significant impact on human energy expenditure.

In certain embodiments, provided are methods for increasing the ratio of brown fat to white fat, or for effecting a change in white adipose tissue to become brown adipose tissue in a subject by administering one or more agents capable of increasing or inducing hypothalamic expression of BDNF. In certain embodiments, provided are methods of treating a subject having obesity. The subject may have, for example, a low ratio of brown adipose tissue to white adipose tissue, diet induced obesity, an obesity related disorder, or a combination thereof. For instance, a subject having a low amount of brown adipose tissue may have an amount by weight of brown adipose tissue of less than about 0.05%, less than about 0.06%, less than about 0.07%, less than about 0.08%, less than about 0.09%, less than about 0.1%, less than about 0.2%, less than about 0.25%, less than about 0.3%, less than about 0.4%, less than about 0.5%, less than about 0.6%, less than about 0.7%, or less than about 0.75% of total body weight. The methods may be effective without a negative impact in blood pressure, heart rate or a combination thereof.

The treatment or prevention of obesity can encompass treatment or prevention of a complication associated with obesity. Complications of obesity include, but are not limited to, hypercholesterolemia, hypertension, dyslipidemia (for example, high total cholesterol or high levels of triglycerides), type 2 diabetes, coronary heart disease, stroke, gallbladder disease, osteoarthritis, sleep apnea and respiratory problems, and some cancers (for example, endometrial, breast, and colon cancer).

As used herein, the term “subject” means a human. In some embodiments, the methods described herein may be used on non-human animals or samples from non-human animals, such as mammals or non-human mammals.

The methods include administering one or more agents capable of increasing or inducing hypothalamic expression of BDNF and/or increasing or inducing BDNF signaling. The agent may increase the level of BDNF expression in the hypothalamus by at least about 5%, at least about 10%, at least about 15%, at least about 20%, at least about 25%, at least about 30%, at least about 40%, at least about 50%, at least about 60% or at least about 70%.

The present invention also relates to methods of preventing weight gain and increasing resistance to diet induced obesity by administering one or more TrkB receptor agonists or nucleotide sequences encoding a TrkB receptor agonist to a subject suffering from diet induced obesity. The TrkB receptor agonist or expression of the TrkB receptor agonist modulates sympathetic nervous system activity, thereby causing a browning of white adipose tissue. More particularly, the TrkB receptor agonist or expression of the TrkB receptor agonist modulates a hypothalamic-adipocyte axis.

Suitable TrkB receptor agonists include, without limitation, peptides, antibodies, and chemical compounds. The peptides can be BDNF, Neurotrophin-4, Neurotropin-3 or a combination thereof. The antibodies can be any antibody that stimulates TrkB receptor activity. The chemical compounds may be 7,8-dihydroxyflavone, N-acetylserotonin, Amitriptyline, 4′-Dimethylamino-7,8-dihydroxyflavone, LM22A-1, LM22A-2, LM22A-3, and LM22A-4. Suitable nucleotide sequences encode, without limitation, BDNF, Neurotrophin-4, Neurotrophin-3, or a combination thereof.

The TrkB receptor agonist or nucleotide sequences encoding the TrkB receptor agonist is administered in an amount sufficient to reduce the ratio of white adipose tissue to brown adipose tissue. Agonists or nucleotide sequences can be administered either systemically by mouth, injection, intramuscular (i.e., im), intravenous (i.e., iv), subcutaneous (i.e., sc), etc., centrally including intrathecally and intracerebroventricularly (i.e., icy), or directly into the brain. Typically, agonists or nucleotide sequences encoding the agonist can be administered via mouth or peripheral injection. The TrkB receptor agonist or expression of the TrkB receptor agonist may reduce the ratio of the white adipose tissue to brown adipose tissue by at least about 5%, at least about 10%, at least about 15%, at least about 20%, at least about 25%, at least about 30%, at least about 40%, at least about 50%, at least about 60% or at least about 70%.

The present invention further provides for methods of preventing weight gain or increasing resistance to diet induced obesity, or a combination thereof by administering one or more beta-3 adrenergic agonists to a subject suffering from diet induced obesity. The subject may have a high ratio of white adipose tissue to brown adipose tissue. The beta-3 adrenergic receptor agonist modulates β-adrenergic signaling, thereby causing a browning of white adipose tissue.

Suitable beta-3 adrenergic receptor agonists include, without limitation, Octopamine, Amibegron (SR-58611A), Solabegron (GW-427,353), Nebivolol, L-796568, CL-316243, LY-368842, and Ro40-2148. The beta-3 adrenergic receptor agonist is administered in an amount sufficient to reduce the ratio of white adipose tissue to brown adipose tissue. The agonists can be administered orally, by injection, or systemically (i.e., peroral) in an amount sufficient to partially or fully activate the beta-3 adrenergic receptor. The beta-3 adrenergic receptor agonist may reduce the ratio of white adipose tissue to brown adipose tissue by at least about 5%, at least about 10%, at least about 15%, at least about 20%, at least about 25%, at least about 30%, at least about 40%, at least about 50%, at least about 60% or at least about 70%.

The agonists described herein may also be employed as therapeutic agents in a subject to increase the amount of brown adipose tissue, decrease the amount of white adipose tissue, increase the ratio of brown adipose tissue to white adipose tissue, decrease the ratio of white adipose tissue to brown adipose tissue, enhance weight loss, prevent weight gain, increase the basal metabolic rate, or any combination thereof. The agonists can be formulated according to known methods to prepare pharmaceutically useful compositions, whereby the agonist is combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants.

The route of administration is in accord with known methods, e.g. oral, injection or infusion by intravenous, intraperitoneal, intracerebral, intramuscular, intraocular, intraarterial or intralesional routes, topical administration, or by sustained release systems.

Dosages and desired drug concentrations of pharmaceutical compositions of the present invention may vary depending on the particular compound and use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

When in vivo administration of an agonist or antibody thereof is employed, normal dosage amounts may vary from about 10 ng/kg to up to 50 mg/kg of mammal body weight or more per day, preferably about 0.01 mg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212, each of which is incorporated by reference in its entirety. It is anticipated that different formulations will be effective for different treatment compounds and different disorders, that administration targeting one organ or tissue, for example, may necessitate delivery in a manner different from that to another organ or tissue.

Where sustained-release administration of an agonist or antibody is desired in a formulation with release characteristics suitable for the treatment of any disease or disorder requiring administration of the agonist or antibody, microencapsulation of the agonist or antibody is contemplated. Microencapsulation of recombinant proteins for sustained release has been successfully performed with human growth hormone (rhGH), interferon-(rhIFN-), interleukin-2, and MN rgp120. Johnson et al., Nat. Med., 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

The sustained-release formulations of these proteins can be developed using poly-lactic-co-glycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

### Materials and Methods

Environmental Enrichment (EE) Protocol with NCD.

Male 3-week-old C57B1/6 mice (from Charles River) were housed in large or EE cages (63 cm×49 cm×44 cm, 5 mice per cage) supplemented with running wheels, tunnels, igloos, huts, retreats, wood toys, a maze, and nesting material in addition to standard lab chow and water. We housed control mice under standard laboratory conditions (5 mice per cage). Mice were fed with normal chow diet (NCD, 11% fat, caloric density 3.4 kcal/g).

Voluntary Running Experiment.

Male 3-week-old C57/BL6 mice were housed in cages with free access to running wheels (Mouse activity wheel with plastic home cage, Med Associates, 5 mice per running cage). Given the difficulty of 3 week old mice to use the activity wheel attached to the home because of the wheel weight and inertia, we put a small plastic running wheel in the home cage for 2 weeks and removed them when the mice were ready to used the attached activity wheel. We fed the mice with NCD.

EE with No Access to Running Wheel Experiment.

We randomly assigned 3-week-old C57/BL6 mice to 4 groups: control housing, EE housing (as described above), EE with no access to running wheel (remove the wheel from EE cage), wheel-running in regular cage (a wheel placed in regular mouse cage). The mice were maintained in the respective housing conditions for 10 weeks.

EE Protocol with HFD.

We randomly assigned 20 C57B1/6 mice to live in EE or control housing as described above for 4 weeks. We switched the diet from NCD to high fat diet (HFD, 45% fat, caloric density 4.73 kcal/g, Research Diets, Inc.) when EE was initiated.

Serum Harvest and Biomarkers Measurement.

We collected blood from the retroorbital sinus. We anesthetized the mice of each group at the same time with ketamine (87 mg/kg)/xylazine (13 mg/kg) followed by blood withdraw. All blood harvesting started at 10:00 am. We prepared serum by allowing the blood to clot for 30 min on ice followed by centrifugation. Serum was at least diluted 1:5 in serum assay diluent and assayed using the following DuoSet ELISA Development System (R&D Systems): mouse IGF-1 and Leptin. Corticosterone was measured using a corticosterone ELISA (ALPCO Diagnostic). Glucose was measured using QuantiChrom Glucose Assay (BioAssay Systems). Total cholesterol was measured by using Cholesterol E test kit (Wako Diagnostics). Thyroid panel (T3, T4, TSH) were measured by ANILYTICS Inc.

AAV Mediated BDNF Overexpression in DIO Mice.

We randomly assigned DIO mice (˜40 g) to receive AAV-BDNF or AAV-YFP. Mice were maintained on HFD throughout the experiment. We generated DIO mice by feeding mice with HFD for 10 weeks until body weights reached 40 g. The obese mice were then randomly assigned to receive AAV-BDNF or AAV-YFP. Mice were anaesthetized with a single dose of ketamine/xylazine (100 mg/kg and 20 mg/kg; i.p.) and secured via ear bars and incisor bar on a Kopf stereotaxic frame. A mid-line incision was made through the scalp to reveal the skull and two small holes were drilled into the skull with a dental drill above the injection sites (−0.8 AP, ±0.3 ML, −5.0 DV; mm from bregma). rAAV vectors (1×10⁹ genomic particles per site) were injected bilaterally into the hypothalamus at a rate of 0.1 μL/min using a 10 μL Hamilton syringe attached to Micro4 Micro Syringe Pump Controller (World Precision Instruments Inc., Sarasota, USA). At the end of infusion, the syringe was slowly removed from the brain and the scalp was sultured. Animals were placed back into a clean cage and carefully monitored post-surgery until fully recovered from anesthesia. We monitored body weight every 5-7 days and recorded the food intake. Mice were maintained on HFD until the end of the study (7 weeks after surgery).

rAAV Vector Construction and Packaging.

The rAAV plasmid contains a vector expression cassette consisting of the CMV enhancer and chicken β-actin (CBA) promoter, woodchuck post-transcriptional regulatory element (WPRE) and bovine growth hormone poly-A flanked by AAV inverted terminal repeats. Human TrkB isoform 1 (TrkB.T1) cDNA was inserted into the multiple cloning sites between the CBA promoter and WPRE sequence. Destabilized YFP were cloned into the rAAV plasmid as controls. rAAV serotype 1 vectors were packaged and purified as described elsewhere (Cao et al., 2004).

AAV Mediated Overexpression of Dominant Negative TrkB.T1 (Eide et al., 1996).

We randomly assigned 8-week old male C57B1/6 mice to receive AAV-TrkB.T1 or AAV-YFP. We injected AAV vectors bilaterally to the hypothalamus (−1.2 AP, ±0.5 ML, −6.2 DV; mm from bregma, 5.0×10⁹ genomic particles per site). We monitored body weight every 5-7 days and recorded the food intake. We kept the mice on NCD till the end of the study 4 weeks after surgery.

AAV-microRNA Experiment.

We randomly assigned 7-week-old C57/BL6 mice to receive AAV-miR-Bdnf (n=12) or AAV-miR-scr (n=12). We injected AAV vectors (1.4×10¹⁰ particles per site) bilaterally into the hypothalamus at the stereotaxic coordinates described above. Ten days after surgery, half of the miR-Bdnf mice and miR-scr. mice were housed in enriched or EE housing with the other half of the groups were maintained in standard housing. After 4 weeks of enriched or EE housing, we dissected fat pads and analyzed the gene expression using qR T-PCR.

Body Weight and Food Consumption.

We maintained the mice on a normal 12 h/12 h light/dark cycle with respective diet (NCD or HFD) and water ad libitum throughout the experiment. Body weight of individual mice was recorded weekly. Food consumption was recorded weekly as the total food consumption of each cage housing 5 mice and represented as the average of food consumption per mouse per day.

MRI Analysis of Fat Mass.

The in vivo MRI with T1-weighted gradient-echo sequence on the mice was performed in the Small Animal Imaging Center of the Ohio State University. Briefly, a Bruker 11.7T NMT system operating at a proton frequency of 500 MHz with a gradient strength of 300 gauss per centimeter was used. Mice were anesthetized with isoflurane and placed in a 30 mm birdcage coil. A coronal spin-echo localizing sequence was used to identify both kidneys. Thirty contiguous, 1 mm thick axial slices spanning from the superior pole of the uppermost kidney to the caudal aspect of the mouse were obtained using a spin-echo sequence. Data analysis was performed by Image J software as described previously (Tang et al., 2002; Xu et al., 2010).

Quantitative RT-PCR.

We dissected brown and white adipose tissues and hypothalamus and isolated total RNA using RNeasy Lipid Kit plus RNase-free DNase treatment (Qiagen). We generated first-strand cDNA using TaqMan Reverse Transcription Reagent (Applied Biosystems) and carried out quantitative PCR using Light Cycler (Roche) with the Power SYBR Green PCR Master Mix (Applied Biosystems). We designed primers to detect the following mouse mRNA: Cartp, Npy, Mc4r, Sgk, Vgf, Insr, Lepr, Ntrk2, Pomc, Mc4r, Trh, Crh, Crhr1, Crhr2, Th, Ucp1, Ucp2, Ucp3, Lep, Adipoq, Ap2, Pparg, Fasn, Agt, Resn, Prdm16, Cidea, Elovls, Ppargc1a, Dio2, Ppard, Adrb1, Adrb2, Adrb3. We calibrated data to endogenous control Actb and quantified the relative gene expression using the equation Tₒ/Rₒ=K×2^{(CT,R-CT,T)}. Tₒ is the initial number of target gene mRNA copies, Rₒ is the initial number of internal control gene mRNA copies, CT,T is the threshold cycle of the target gene, CT,R is the threshold cycle of the internal control gene and K is a constant.

BDNF Heterozygous Mice.

We used male BDNF+/− mice provided by Dr. F. Lee of Weill Medical College of Cornell University to breed a BDNF+/− colony. We dissected brown and white fat pads of male BDNF heterozygous mice (n=4) and age matched C57BL6 wild type littermates (n=4) at the age of 2 months.

Immunohistochemistry.

We cut paraffin-embedded sections (4 μm) of adipose tissues and subjected the sections to citrate-based antigen retrieval following by incubations with antibody against UCP1 (Abcam). The sections were visualized with DAB and counterstained with hematoxylin.

TUNEL Assay.

Adipose tissue was snap-frozen and stored at −80° C. till use. 10 μm sections were cut and fixed with 4% paraformaldehyde. TUNEL assay was performed using in situ Cell Death Detection kit (Roche) following the manufacturer's instructions. The intestinal section from the same animal was used as positive control.

Propranolol Experiment.

We randomly assigned 40 C57BL/6 mice, 3 weeks of age, to live in enriched or control cages supplied with propranolol in drinking water (0.5 g/L). We collected blood 3 weeks after enrichment and dissected fat pads after 5 weeks enrichment.

β-Blockade Using Osmotic Minipump.

We bilaterally injected AAV-BDNF (n=15) or empty AAV vectors (n=10) to the hypothalamus of 25 C57BL/6 mice, 8 weeks of age as described above. Eight weeks after AAV injection, mice of each vector injection were randomly divided to two subgroups. Osmotic minipumps (Alzet micro-osmotic pump model 1002) containing the combination of β blockers propranolol (Sigma, 2 mg/kg/d for 14 days) (Thaker et al., 2006) and SR59230A (Sigma, 1 mg/kg/d for 14 days) (Frye et al., 2009) or vehicle were implanted s.c. on the back. 14 days after pump implantation, fat depots were dissected and adipose tissue gene expression was measured by qRT-PCR.

Norepinephrine Measurement.

We used the Norepinephrine ELISA kit (Labor Diagnostika Nord GmbH) to measure norepinephrine in serum and fat lysates. We sonicated the tissues in lysis buffer (20 mM TrisCl, 1 mM EDTA) for the assay and calibrated to the tissue weight.

Basal Resting Oxygen Consumption.

Basal/resting metabolic rate (RMR) was measured in mice after 5 weeks enrichment or control housing. Oxygen consumption was measured by indirect calorimetry (Columbus Instruments) in conscious animals at 30° C. during 3 hr in the light period in the absence of food and water. RMR was defined as the average of the three lowest reading.

Norepinephrine Stimulation In Vivo.

To measure the norepinephrine-induced gene expression changes in white fat, the mice after 6 weeks enrichment or control housing were anesthetized with pentobarbital (90 mg/kg, i.p.) and injected with 0.3 mg/kg norepinephrine (s.c.). The mice were maintained at 30° C. throughout the experiment. Fat pads were dissected 4 h after norepinephrine injection. qRT-PCR was used to profile gene expression in white fat.

Adipose Tissue Oxygen Consumption.

Fat pads were dissected, cut to small pieces and placed to a BD Oxygen Biosensor System plate (BD Bioscience) in triplicate, 150 μl DMEM+1% BSA/well. The plate was sealed and read on a SPECTRAFluor Plus microplate spectrophotometer (Tecan) at 5 min interval for 60 min at an excitation wavelength of 485 nm and emission wavelength of 595 nm. Brown adipose tissue, epididymal white adipose tissue, inguinal white adipose tissue and retroperitoneal white adipose tissue of each animal were measured, 3 mice per group. Normalized relative fluorescence (NRF) was calculated according to manufacturer's instruction and was calibrated to fat tissue weight.

Mitochondrial DNA measurement. Total DNA was isolated using DNeasy Blood & Tissue Kit (Qiagen). Mitochondrial mass was determined by measuring mitochondrial DNA-encoded cytochrome c oxidase subunit 1 (Cox I) by qPCR. Cox I levels were normalized to Bdnf encoded by nuclear DNA.

Western Blot.

We used ALLPrep DNA/RNA mini kit (Qiagen) to simultaneously purify genomic DNA and total RNA. Proteins were precipitated from the flow-through using acetone according to manufacturer's protocol. Rabbit anti UCP1 (Abcam, 1:4000), mouse anti HSP60 (Chemicon, 1:500), rabbit anti β3 adrenergic receptor (Chemicon, 1:500), rabbit anti PGC-1α (Cell Signaling, 1:1000), and rabbit anti pan actin (Cell signaling, 1:1000) were used in western blot analysis.

Statistical Analysis.

Data are expressed as mean±s.e.m. We used JMP software to analyze the following: repeated measures ANOVA for food intake and oxygen consumption; one-way ANOVA for serum biomarker measurements, body weight and adipose tissue weight, body temperature, quantitative RT-PCR data; regression for adipose tissue weight-gene expression correlation.

### Example 2

### EE Decrease Adiposity

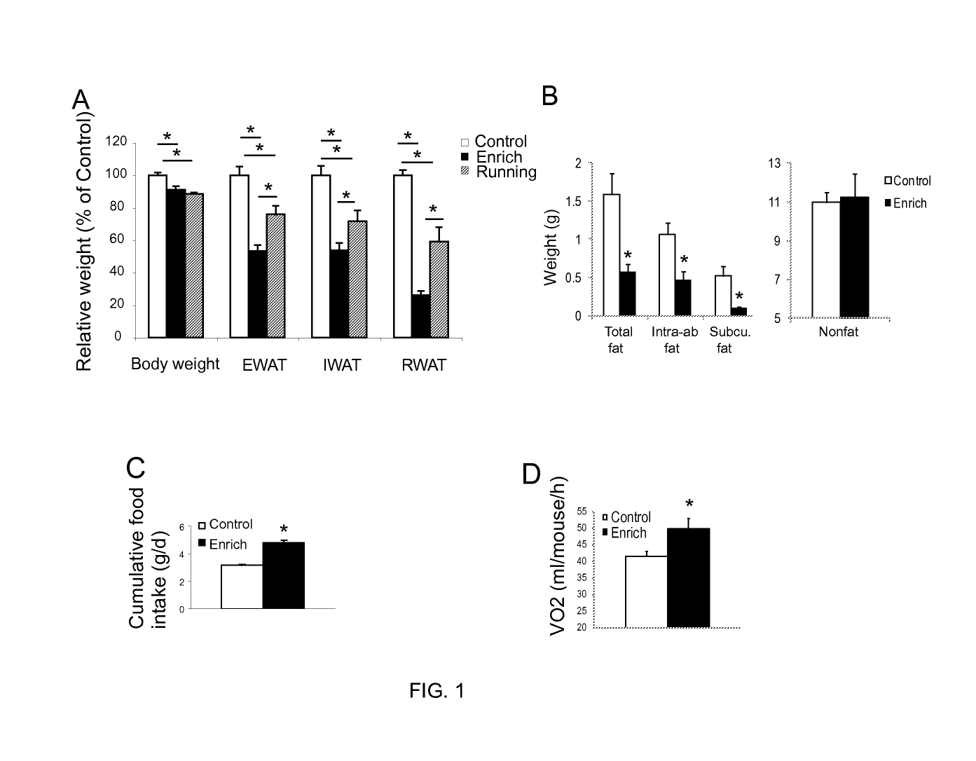

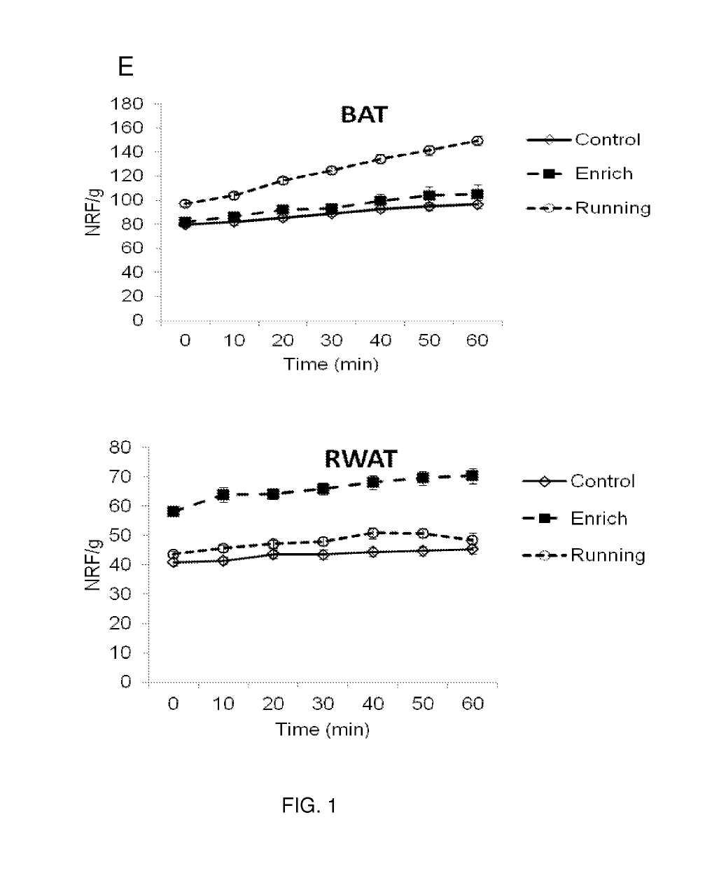

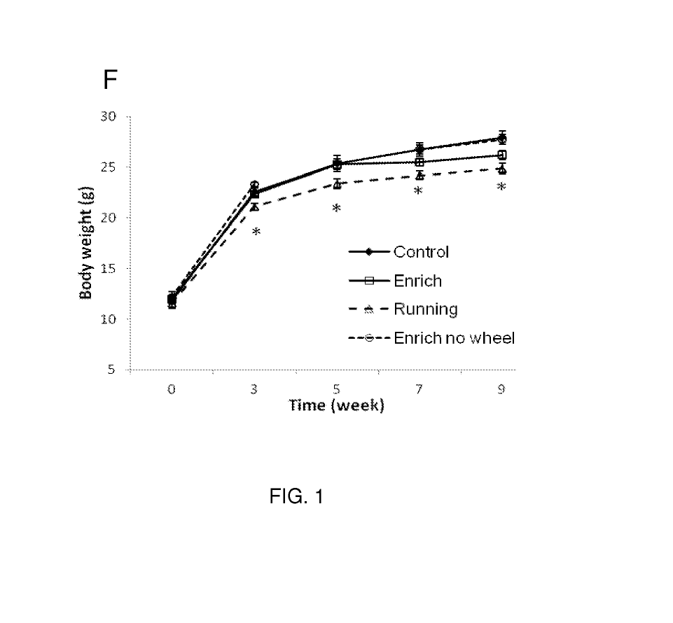

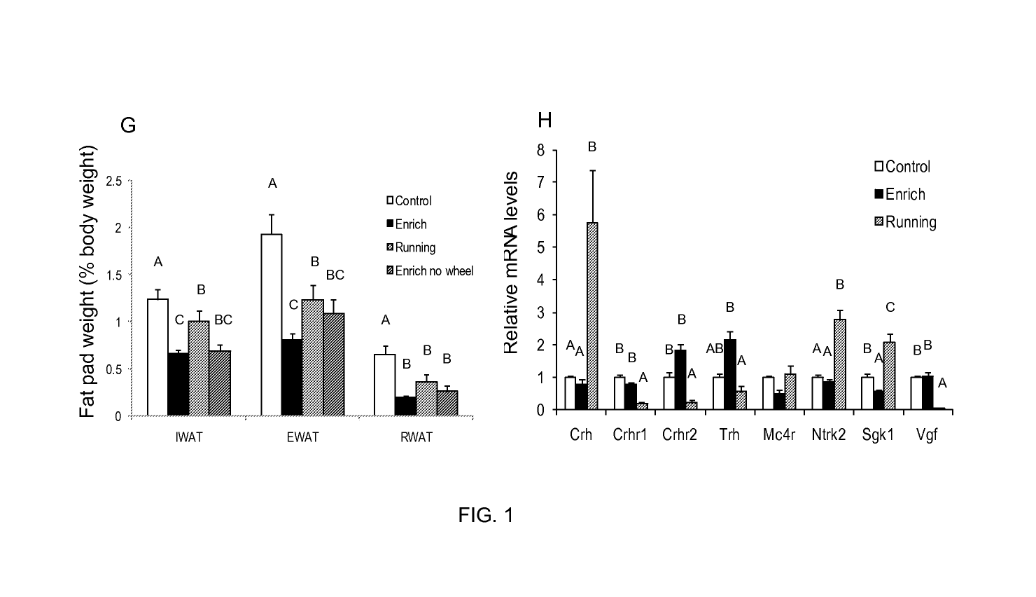

C57BL/6 mice were randomly assigned to live in either grouped control housing or EE with larger space, running wheels and regularly changed toys and mazes under an ambient temperature of 22° C. Both control and EE animals had free access to normal chow diet (NCD) and water. After 4 weeks EE, the EE mice had slightly lower body weights than control mice and a marked reduction in white adipose tissue mass (FIG. 1A). All of the white adipose tissue pad weights were markedly decreased: inguinal white adipose tissue by 46.3±4.8%, epididymal white adipose tissue by 46. 7±3.7%, and retroperitoneal white adipose tissue by 73.7±2.5%. Magnetic resonance imaging (MRI) showed 49.3±12.8% decrease of abdominal fat mass (FIG. 1B). Since physical exercise decreases body fat, we subjected another group of mice to voluntary wheel running for 4 weeks. Running decreased body weight similarly to EE mice (FIG. 1A). However, the reduction in white adipose tissue in runners was significantly less than that observed with EE (FIG. 1A, P<0.05). Both EE and running increased gastrocnemius mass by 22.7±3.5% and 12.8±2.5%, respectively. We traced the traveling distance of EE mice plus the wheel running distance excluding activity within the feeding cage (the regular mouse cage) (Cao et al., 2010). The total distance traveled by the EE mice was 0.86 km/d more than control mice but approximately 66% lower than runner's average traveling distance 2 km/d/mouse indicating the further reduction of adiposity was not due to greater overall motor activity in EE.

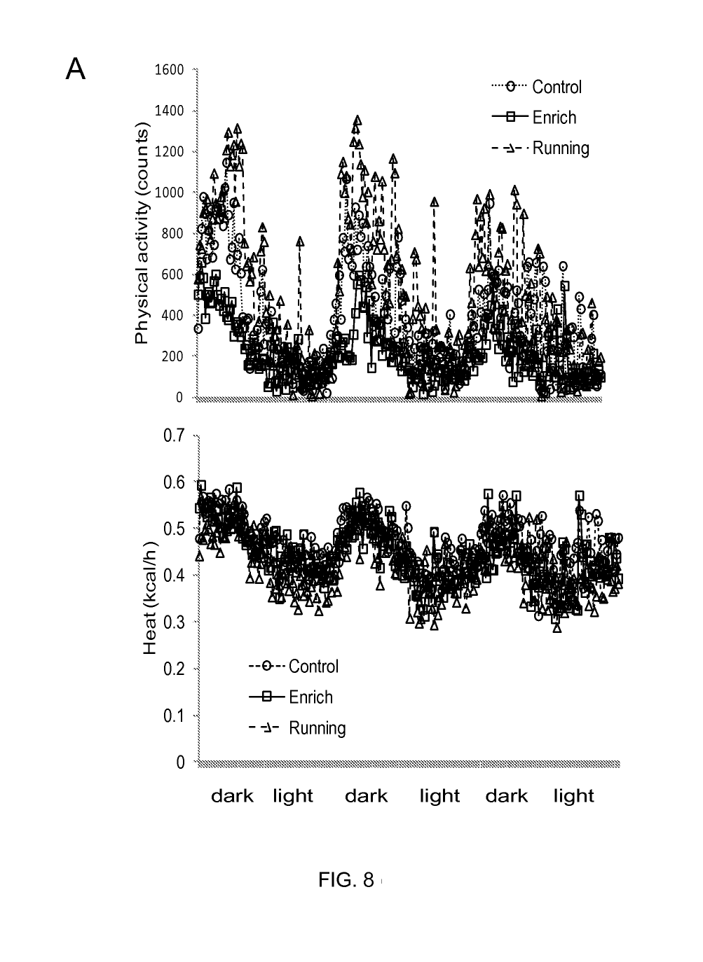

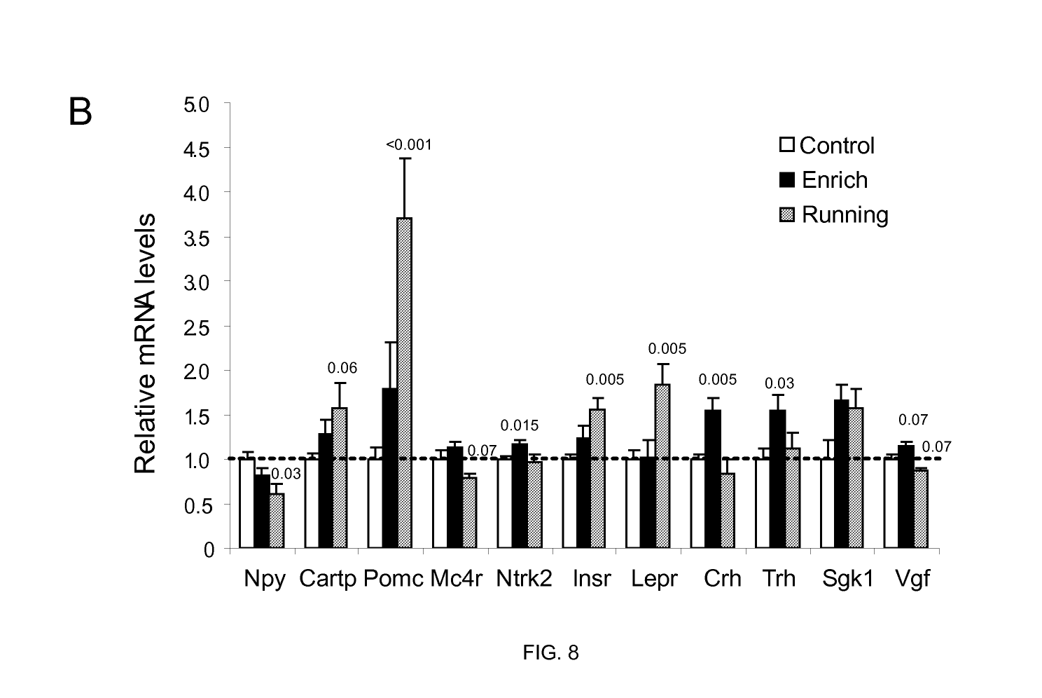

We measured the whole-body metabolism of mice after 5 weeks EE using the CLAMS (Comprehensive Lab Animal Monitoring System) for 3 days at room temperature. No difference in oxygen consumption was found in the EE mice compared to the control mice. However, the EE mice, upon removal from their complex environment, showed significantly lower physical activity in the metabolic chambers (FIG. 8, P<0.05). The change of environment from EE to single housing in the metabolic chamber and the impact on metabolism resulting from the coping behavior complicates the evaluation of energy expenditure. Therefore the data from the CLAMS may not properly represent the energy expenditure in the EE. In contrast to EE, wheel running mice showed higher physical activity in the metabolic chambers compared to control mice, yet no significant increase in oxygen consumption was observed (FIGS. 8A,B), further demonstrating the limitation of CLAMS in the setting of acute environmental changes. We then used an alternative approach to measure metabolism for 3 hours starting immediately upon removal from the EE cage, the EE mice showed increased basal resting oxygen consumption at thermoneutrality (FIG. 1D) (Feldmann et al., 2009). To supplement the whole body metabolism measurement, we measured the oxygen consumption of dissected fat depots ex vivo. The EE mice showed increased oxygen consumption in retroperitoneal white adipose tissue whereas running mice showed higher oxygen consumption in brown adipose tissue (FIG. IE). In addition the EE mice showed increased food intake (FIG. 1 C) indicating that elevated energy expenditure rather than appetite suppression underlies the lean phenotype.

To further investigate the extent to which physical activity accounts for the lean phenotype induced by EE, we analyzed another group of mice housed in the EE cage with the running wheel removed. In addition, instead of being housed in the running wheel cages equipped to measure running distance in the previous experiment, the voluntary running group in this experiment was housed in the regular control cages with access to the same wheels as the EE mice. We monitored body weight closely over 9 weeks. In contrast to the 4 weeks EE with minimal handling, the runners were the only group that showed a significant decrease in body weight while the body weight of EE mice with no access to running wheels was identical to the control mice (FIG. 1F). However the standard EE mice showed the most marked reduction in adiposity with epididymal white adipose tissue, retroperitoneal white adipose tissue, and inguinal white adipose tissue decreased by 58.4±3.6%, 70.0±2.5%, and 46.5±3.6%, respectively (FIG. 1G). The EE no wheel mice showed a trend towards greater inguinal white adipose tissue and epididymal white adipose tissue mass reduction than the wheel running mice when fat mass was standardized to body weight (FIG. 1G) suggesting that wheel running was not essential for the EE-induced fat mass reduction.

In order to investigate the potential mechanisms differentiating EE- and running-induced lean phenotypes, we profiled gene expression in the hypothalamus, one of the brain regions involved in energy homeostasis. EE and running led to multiple changes in the expression of genes involved in energy balance but displayed two distinctive profiles in the whole hypothalamus dissections after 4 weeks EE or running (FIG. 8B). The hypothalamus contains a number of discrete nuclei including the arcuate (ARC), paraventricular (PVH), ventromedial (VMH), dorsomedial (DMH), and lateral hypothalamic area (LH). We previously observed differential gene expression profiles in the ARC, which has traditionally been considered a primary site for the central action of leptin on energy homeostasis (Stephens et al., 1995), of the EE mice compared to the running mice after 4 weeks of respective housing (Cao et al., 2010). Here we analyzed gene expression in the PVH, which was recently shown to be not simply downstream of the ARC, but an additional primary site for a multinodalleptin/MC4R system regulating energy homeostasis (Ghamari-Langroudi et al., 2011). After 10 weeks EE or running we observed two qualitatively distinct patterns in the PVH (FIG. 1H). Running led to the induction of corticotrophin-releasing hormone (CRH) accompanied by the significant downregulation of its two receptors Crhr1 and Crhr2. EE showed a trend of up regulation of thyrotropin-releasing hormone (TRH) in contrast to the trend of downregulation in the running mice (FIG. 1H) suggesting that the EE and running reduce adiposity via distinct mechanisms. Thyroid hormones increase energy expenditure via a process termed “thyroid thermogenesis” (Cannon and Nedergaard, 2010) and recent findings suggest thyroid hormones affect the hypothalamus and subsequently activate brown adipose tissue leading to increased energy expenditure (Lopez et al., 2010). EE upregulated Trh expression in the hypothalamus, however hyperthyroidism was not observed in mice after 9 weeks EE (serum T3: Control 0.746±0.025 ng/ml, EE 0.649±0.036 ng/ml, P=0.062; T4: Control 4.52±0.18 μg/dl, EE 3.15±0.38 μg/dl, P=0.01; TSH: Control 0.112±0.024 μg/ml, EE 0.095±0.024 μg/ml, P=0.64).

### Example 3

### EE Induces Brown Fat Molecular Phenotype in White Adipose Tissue

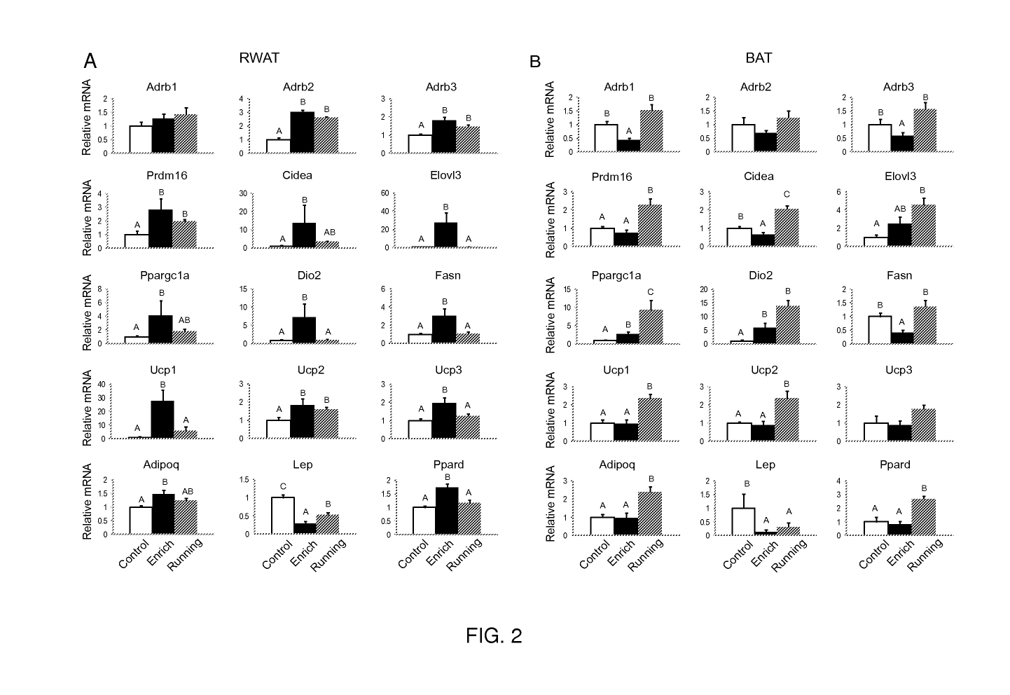

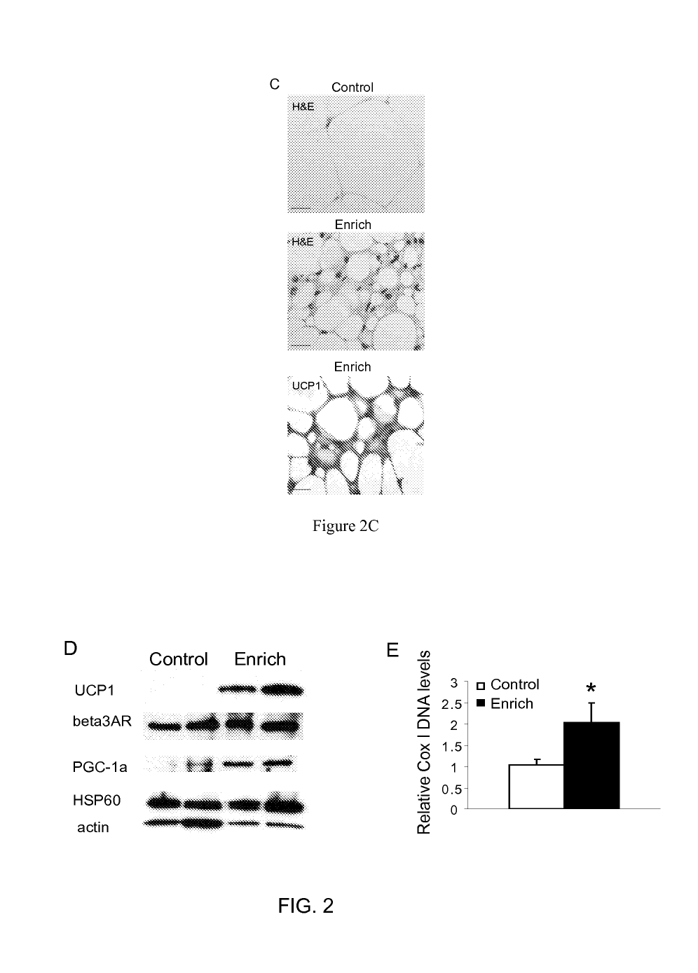

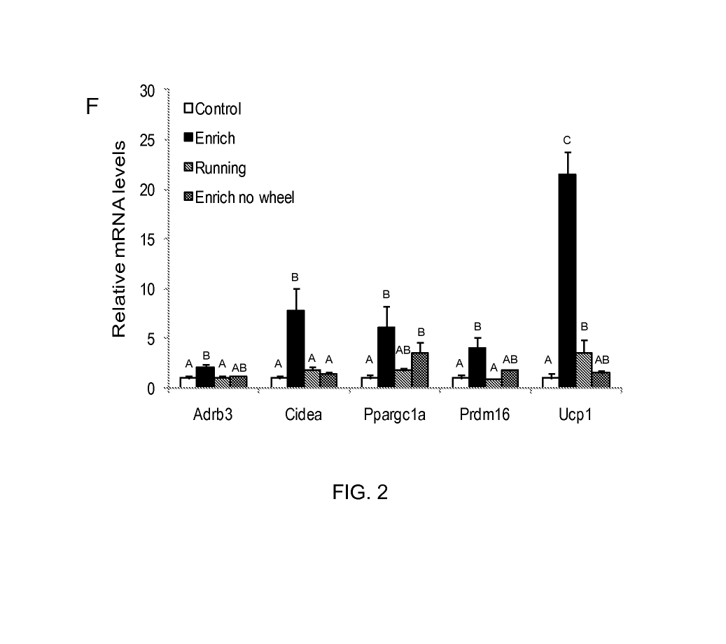

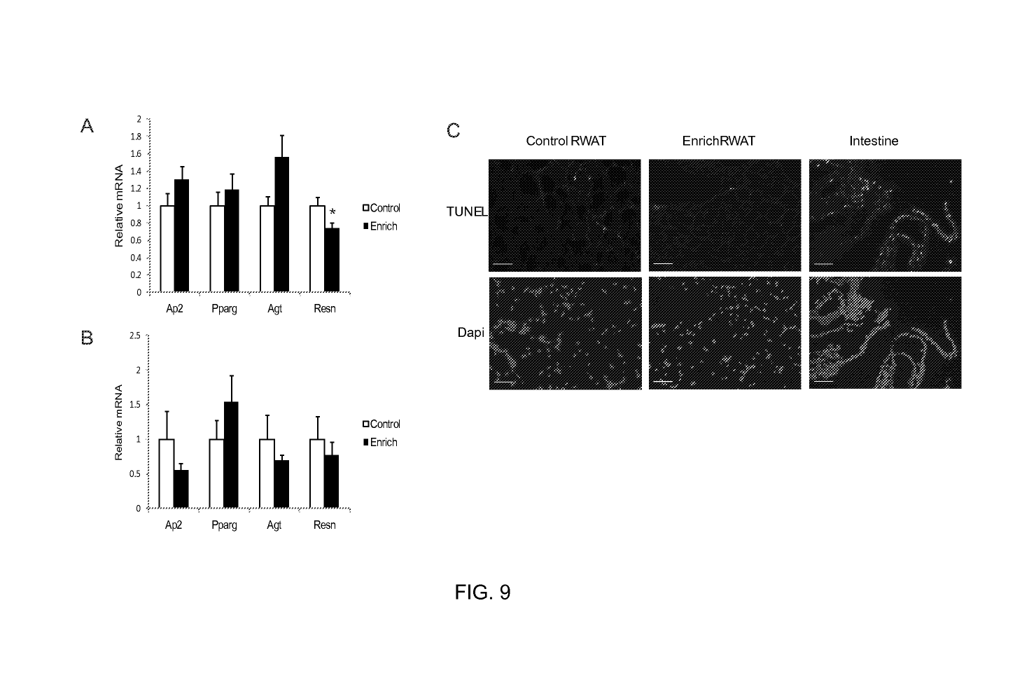

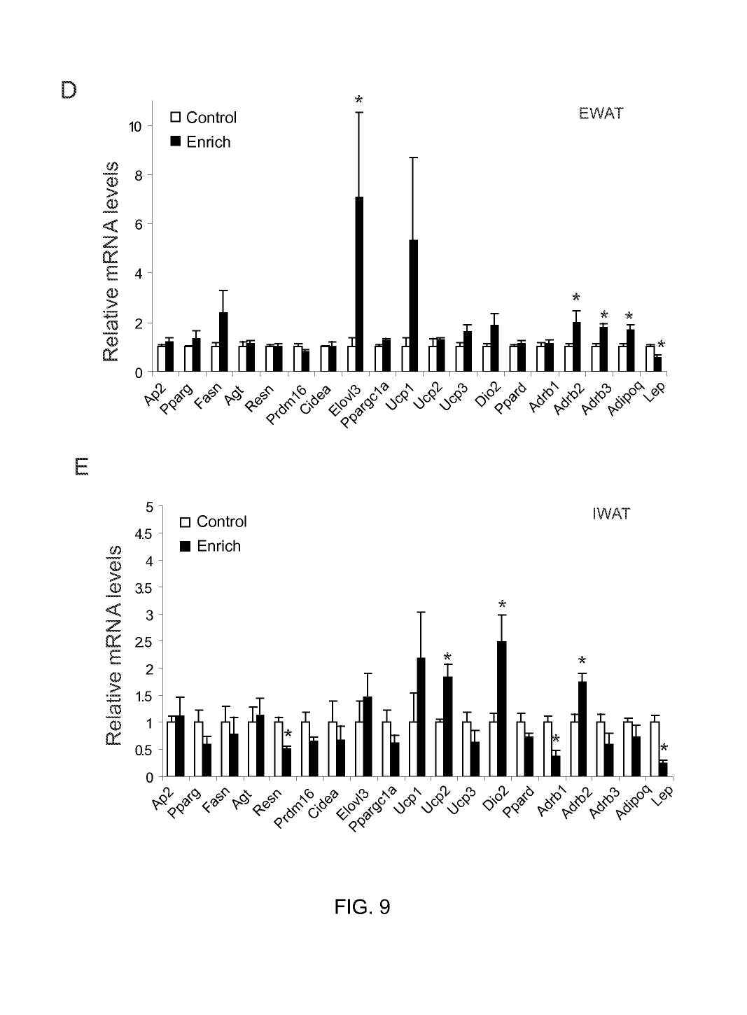

Brown adipose tissue and white adipose tissue perform opposing functions with white adipose tissue accumulating surplus energy while brown adipose tissue dissipating energy as heat. We examined the impact of EE on brown adipose tissue and white adipose tissue gene expression profiles by quantitative RT-PCR after 4 weeks of EE (FIGS. 2A, B, FIGS. 9A, B). Limited gene expression changes were observed in brown adipose tissue with 6 out of the 19 genes profiled showing significant change (FIG. 2B, FIG. 9B). In contrast, retroperitoneal white adipose tissue was far more responsive to the EE with 15 out of the 19 genes profiled showing altered expression (FIG. 2A, FIG. 9A). Prdm16 (PR-domain-containing 16), which has been identified as a genetic switch determining the formation and function of brown adipocytes (Seale et al., 2008) was significantly upregulated by 2.8±0.8 fold in retroperitoneal white adipose tissue. The induction of Prdm16 was accompanied by the robust induction of a brown adipose tissue molecular signature including Cidea (13.6±1.0 fold), Elovl3 (27.4±11.2 fold), Ucp1 and Ppargc1a (encoding PGC-1α), all of which are brown adipose tissue selective markers and positively regulated by Prdm16 (Seale et al., 2008). PGC-1α has been shown to switch cells from energy storage to energy expenditure phenotype by inducing mitochondrial biogenesis and genes involved in thermogenesis (Puigserver et al., 1998). Data from knockout mouse strains suggest that several transcriptional regulators, such as RIP140 (Leonardsson et al., 2004), SRC2 (Picard et al., 2002), Rb (Hansen et al., 2004), and Twist1 (Pan et al., 2009), control brown adipocyte development and function, at least in part, through regulating the transcriptional activity or gene expression of PGC-1α. This transcriptional co-activator was upregulated 4.1±2.1 fold in retroperitoneal white adipose tissue of EE mice. Brown adipose tissue dissipates energy via releasing chemical energy from mitochondria in the form of heat. This phenomenon is primarily mediated by UCP 1 (Ucp1) which is a specific brown adipose tissue marker (Nicholls and Locke, 1984). Ucp1 was increased 27.7±8.0 fold in retroperitoneal white adipose tissue of EE mice. Other uncoupling proteins with a role in protection against oxidative damage, Ucp2 and Ucp3, were also upregulated. In addition, Dio2 (type 25′ deiodinase) and Ppard, both involved in oxidative metabolism, were upregulated by 7.3±3.7 fold and 1.7±0.1 fold respectively. β-adrenergic signaling plays an important role in the activation of brown adipose tissue in response to cold and the regulation of adiposity (Bachman et al., 2002; Himms-Hagen et al., 1994; Xue et al., 2007). Both Adrb2 and Adrb3 encoding β-adrenergic receptor (AR) 2 and 3 were upregulated in retroperitoneal white adipose tissue of EE mice (FIG. 2A) while Adrb1 and Adrb3 were downregulated in brown adipose tissue (FIG. 2B). In contrast to the strong induction of the brown adipocyte gene program in retroperitoneal white adipose tissue, EE suppressed the expression of the white fat-enriched gene Resn (resistin) and had no impact on adipocyte markers shared by both brown and white fat such as adipogenic transcription factor Pparg and adipocyte differentiation marker Ap2 (Kajimura et al., 2008) (FIG. 9A). H&E staining showed that the retroperitoneal white adipose tissue adipocytes of EE mice were smaller than those in the control mice (FIG. 2C). No increase in apoptosis measured by TUNEL was observed in EE mice (FIG. 9C). Moreover, pockets of cells with the multilocular morphology characteristic of brown adipocytes (Tsukiyama-Kohara et al., 2001) were observed in retroperitoneal white adipose tissue (FIG. 2C) Immunohistochemical staining showed substantially higher level of UCP1 protein not only in the cells with typical brown fat morphology but also in some cells with unilocular white fat morphology surrounding the pocket of brown fat-like cells (FIG. 2C) (Tsukiyama-Kohara et al., 2001). Western blot showed marked increase of UCP1, mitochondrial protein HSP60, β3AR, and PGC-1α levels in retroperitoneal white adipose tissue of EE mice (FIG. 2D) consistent with the upregulation of mRNAs. Moreover, the mitochondrial DNA content of EE retroperitoneal white adipose tissue was increased by two-fold, indicating enhanced mitochondrial biogenesis (FIG. 2E) (Xue et al., 2007). The other two white fat pads epididymal white adipose tissue (FIG. S2D) and inguinal white adipose tissue (FIG. 9E) showed fewer genetic changes compared to retroperitoneal white adipose tissue. However, expression of the major adipokine, leptin (Lep), was highly suppressed in all fat pads (FIGS. 2A, 2B, FIGS. 9D, E) consistent with the observed sharp drop in leptin serum levels (64.3±4.0% decrease).

Although efficient in reducing adiposity, voluntary wheel running for 4 weeks induced different gene expression profiles in brown adipose tissue and retroperitoneal white adipose tissue compared to EE. Running led to more robust changes in brown adipose tissue than EE (FIG. 2B). However less brown adipose tissue selective genes were induced in retroperitoneal white adipose tissue of the running mice (FIG. 2A). We analyzed the retroperitoneal white adipose tissue gene expression profiles after 10 weeks EE, wheel-running, and EE without wheel. The standard EE induced the most robust brown adipose tissue gene program in retroperitoneal white adipose tissue while the effects of wheel-running and EE without wheel were not additive (FIG. 2F). The molecular signatures in fat induced by the different environmental interventions suggest distinctive molecular mechanisms underlying the reduction in adiposity in response to EE and running.

### Example 4

### EE Inhibits DIO

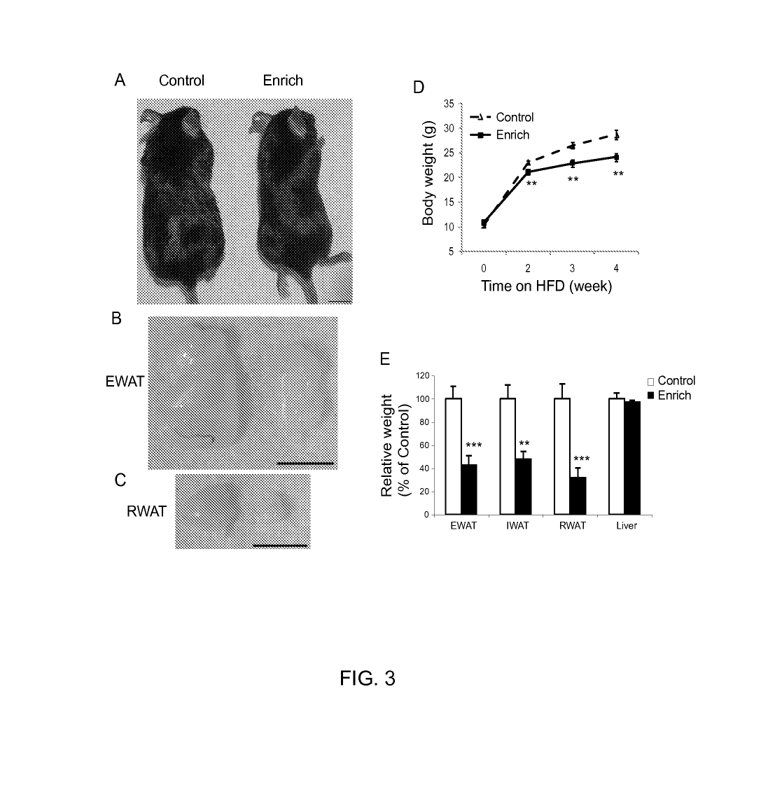

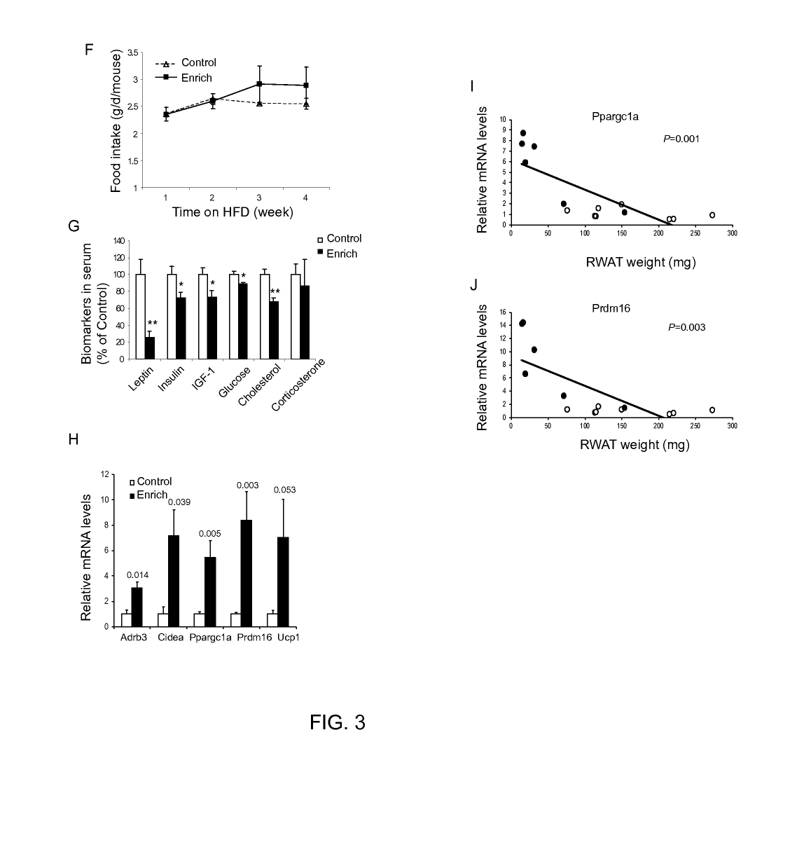

EE induced a “browning” molecular signature in white fat suggesting that an individual's interaction with its immediate environment could switch a white fat energy storage phenotype to a brown fat-like energy expenditure phenotype and regulate adiposity. To test this hypothesis, we investigated whether this transformation could help animals resist DIO. The chow was changed to a high fat diet (HFD, 45% fat, caloric density 4.73 kcal/g) immediately after mice were randomly assigned to live in EE or control housing. After 4 weeks HFD feeding the EE mice gained less weight (71.3±2.7% of control mice weight gain, FIGS. 3A, 3D) and remained lean with significantly smaller fat pads (P<0.01, FIGS. 3B, 3C, 3E). No change in food intake was observed (FIG. 3F). The body temperature of EE mice was increased (EE: 34.86±0.20° C. vs. Control: 34.17±0.14° C., P=0.01) suggesting that elevated energy expenditure, not appetite suppression, led to the resistance to obesity. Moreover, EE prevented DIO associated hyperinsulinemia, hyperleptinemia, hyperglycemia and dyslipidemia (FIG. 3G). Similar to NCD, EE also induced the brown adipose tissue molecular signature in DIO mice (FIG. 3H) and the levels of Ppargc1a and Prdm16 were inversely correlated with retroperitoneal white adipose tissue mass (FIGS. 3I, 3J, P<0.0001), consistent with a robust functional effect of these molecular changes.

### Example 5

### Long-Term EE Leads to Stronger White Adipose Tissue “Browning”

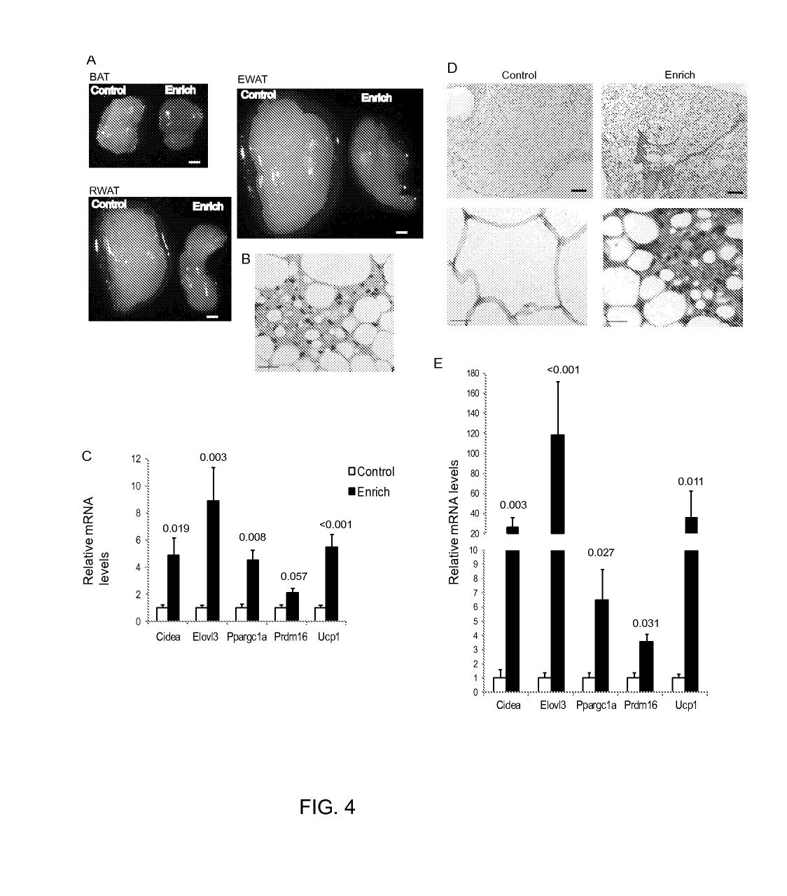

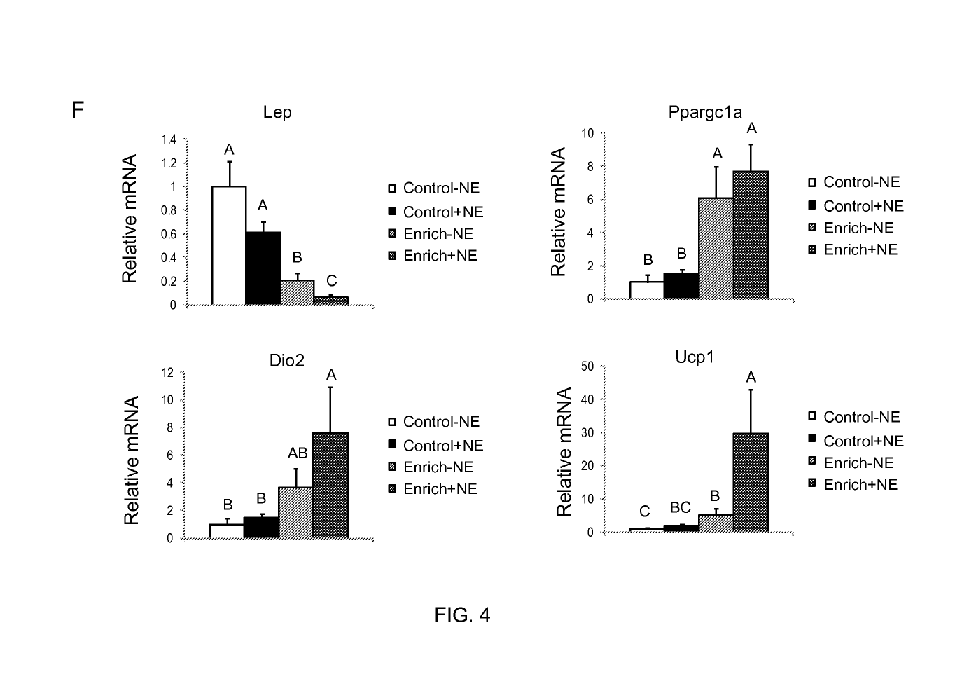

EE of 4 weeks was sufficient to induce white adipose tissue “browning” and resistance to DIO. We then investigated the long-term effect of EE for 3 months. In EE mice fed on NCD, clear macroscopic changes in fat pads were visible to the eye, with white adipose tissue turning brown and brown adipose tissue going even darker (FIG. 4A). Brown adipocyte-like cells were found in epididymal white adipose tissue of long-term EE mice (FIG. 4B) which was rare in short-term EE mice. The expressions of brown fat gene markers were further upregulated in epididymal white adipose tissue (FIG. 4C, FIG. S4A). In contrast to pockets of brown adipocyte-like cells with UCP1 expression found in retroperitoneal white adipose tissue of short-term EE mice, widespread and stronger staining of UCP1 was observed in long-term EE mice (FIG. 4D) associated with more robust induction of the brown fat gene program (FIG. 4E). For example, Elovl3 was upregulated by 118±53.4 fold after 3 months EE while it was induced by 27.4±11.2 fold after 4 weeks EE. Despite their different anatomy and function, brown and white adipocytes are found together in fat depots and the white adipose tissue/brown adipose tissue ratio varies with genetic background, sex, age, nutritional status, and environmental conditions supporting the concept of an adipose organ, a multi-depot organ consisting of two tissues displaying considerable plasticity (Frontini and Cinti, 2010). The darker brown adipose tissue depot observed in long-term EE may indicate a shift towards brown adipocytes in this depot and/or enhanced thermogenic activity that requires further investigation.

### Example 6

### EE Enhance White Adipose Tissue Response to Sympathetic Stimulation

The thermogenic activity of brown adipose tissue is dependent on intact sympathetic stimulation (Landsberg and Young, 1984) and SNS stimulation is essential to induce brown fat-like cells in white adipose tissue depots (Cannon and Nedergaard, 2004). Brown adipose tissue is profusely innervated by sympathetic nerve terminals with norepinepherine (NE) acting via β-ARs. White adipose tissue is also innervated although to a lesser degree (Slavin and Ballard, 1978; Youngstrom and Bartness, 1995). In mice fed on a NCD, EE led to approximate 2 fold increase of NE in white adipose tissue (EE 50.0±6.2 pg/mg vs. Control 25.1±3.8 pg/mg, P=0.012) while no significant increase in serum, muscle or brown adipose tissue. Although NE content per se is not an index of NE release or sympathetic tone, the coordinated upregulation of β-ARs and NE levels in white adipose tissue is consistent with a change in β-AR signaling and might partially explain the selective regulation of white adipose tissue by EE.

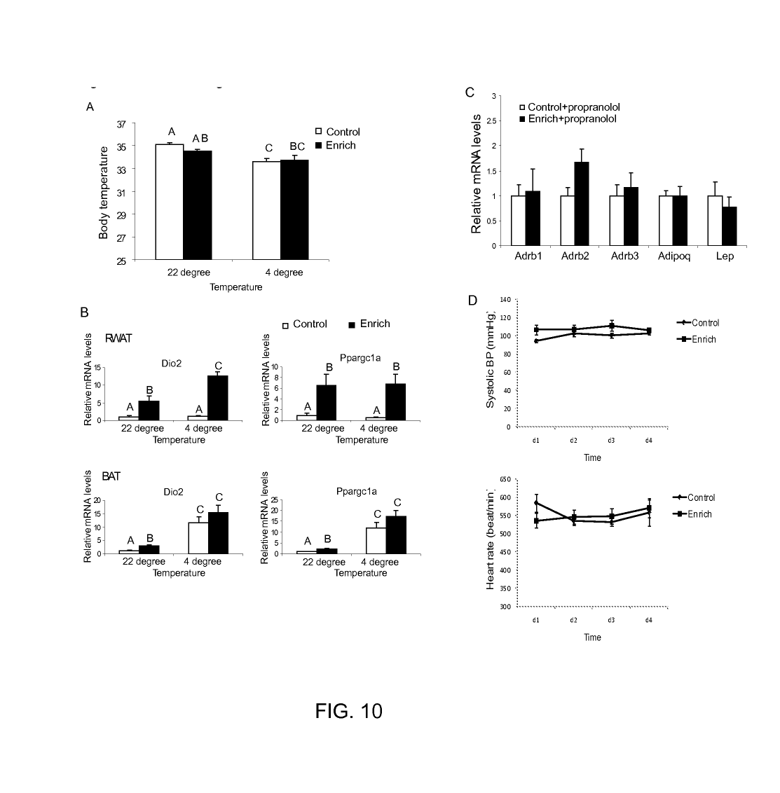

Given the fact that EE led to a marked reduction of adiposity, we sought to investigate whether this reduction would influence the animals' response to cold. Mice of 3 months EE or control housing were randomized to be maintained at 4° C. or 22° C. After 3 hours acute cold exposure, the EE mice maintained a similar body temperature as control mice (FIG. 10A). We examined the expression of genes involved in thermogenesis and known to be activated in response to cold in both brown adipose tissue and white adipose tissue. Both Dio2 and Ppargc1a expression levels were higher in brown adipose tissue of EE mice than control mice at 22° C. and their expression was upregulated after cold exposure (FIG. 10B). Both genes were also upregulated in response to cold in control mice brown adipose tissue (FIG. 10B). However, in retroperitoneal white adipose tissue of control mice, neither Dio2 nor Ppargc1a were changed after cold exposure consistent with the lack of role of white adipose tissue in acute cold response (FIG. 10B). In contrast, Dio2 expression in retroperitoneal white adipose tissue of EE mice was 5.43±1.47 fold higher than control mice at 22° C., and its expression levels was further upregulated by another 2.55±0.22 fold in response to cold (FIG. 10B) suggesting an enhanced molecular response of the EE white adipose tissue to acute cold. We further examined the sensitivity of retroperitoneal white adipose tissue of EE mice to NE stimulation at thermoneutrality of 30° C. Mice of 10 weeks EE were injected with NE (0.3 mg/kg, s.c.) under anesthesia and the expression of genes known to be regulated by NE (Petrovic et al., 2010) was examined in retroperitoneal white adipose tissue 4 hours after NE injection. The low dose NE failed to induce significant changes in control retroperitoneal white adipose tissue (FIG. 4F). In contrast, retroperitoneal white adipose tissue of EE mice were highly responsive to acute low dose NE stimulation. Lep which was sharply downregulated in EE retroperitoneal white adipose tissue compared to control mice was further reduced significantly after NE injection. Ucp1 was highly increased by NE stimulation in the EE retroperitoneal white adipose tissue (FIG. 4F).

We then investigated the role of the global sympathetic drive in mediating EE regulation of white adipose tissue by using the β-blocker propranolol. Mice receiving propranolol in their drinking water were randomly assigned to live in EE or control housing for 5 weeks. Propranolol efficiently blocked the molecular features associated with EE (FIG. 10C) suggesting the essential involvement of the SNS.

### Example 7

### Hypothalamic-Adipocyte Axis Mediates EE-Induced White Adipose Tissue “Browning”

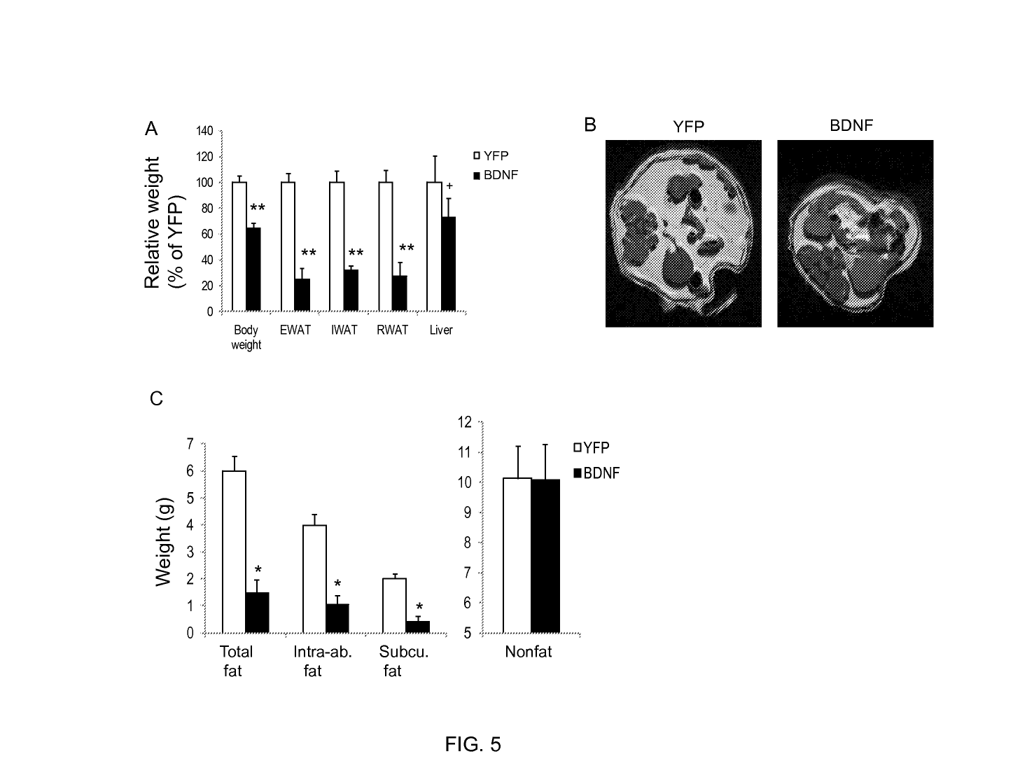

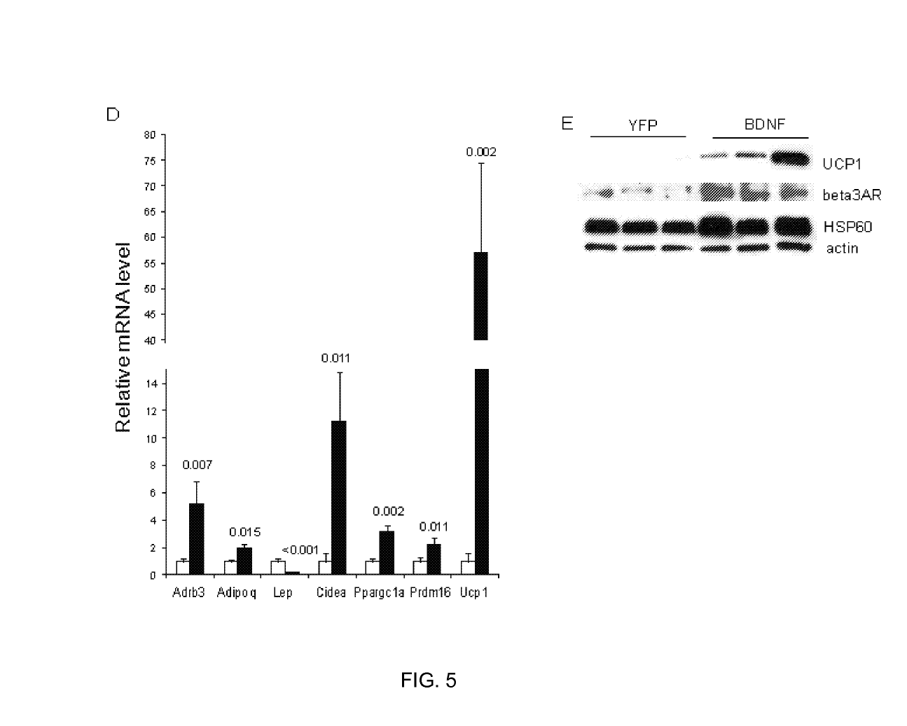

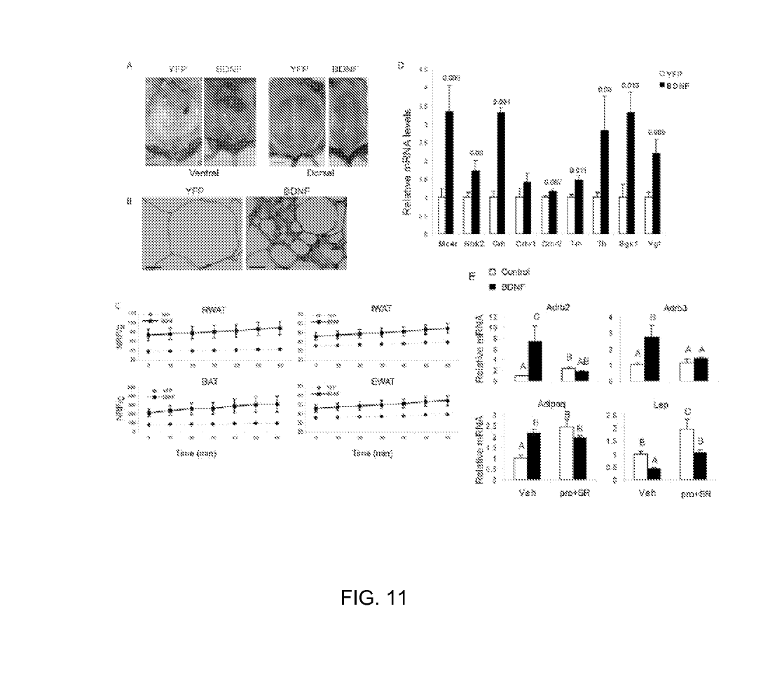

EE inhibits tumor growth. The anticancer effect is mediated by hypothalamic brain-derived neurotrophic factor (BDNF) via activation of the hypothalamic-sympathoneural-adipocyte (HSA) axis (Cao et al., 2010). Using laser capture microdissection, we found a consistent upregulation in hypothalamic BDNF in EE mice but not in voluntary running (Cao et al., 2010). BDNF has recently been identified as a key element in energy homeostasis (Lyons et al., 1999; Rios et al., 2001; Xu et al., 2003). We further investigated the role of BDNF in EE-induced “browning” of white adipose tissue. A rAAV vector expressing the human BDNF gene was injected to the hypothalamus of DIO mice with YFP as a control (Cao et al., 2009). The transgene expression level, location (ARC and VMH) and duration were similar to the previous studies (Cao et al., 2009; Cao et al., 2010). BDNF overexpression led to marked weight loss and fat depletion (FIGS. 5A-C, FIG. S4A) which reproduced the impact of EE on DIO mice but to a greater degree (FIG. 3E). Similarly, BDNF overexpression resulted in the EE-associated molecular features, e.g. upregulation of β-ARs and brown adipose tissue markers in retroperitoneal white adipose tissue (FIG. 5D). Consistent with the marked increase in proteins involved in thermogenesis and mitochondrial function, e.g. UCP1 and HSP60 (FIG. 5E, FIG. 11B), all dissected fat depots of BDNF mice showed substantially higher oxygen consumption ex vivo (FIG. 11C). The BDNF overexpressing mice showed similar gene expression changes to EE mice in the whole hypothalamus dissections (FIG. 11D). We further investigated whether β-blockade could attenuate hypothalamic BDNF's regulation of WAT. AAV-BDNF or empty viral vectors were injected to the hypothalamus bilaterally. Eight weeks after viral vector injection when both the change in body weight (BDNF: −3.8±0.8 g vs. Empty vector: +2.9±0.6 g, P<0.001) as well as cumulative food intake (BDNF: 4.77±0.13 g/d vs. Empty vector: 4.04±0.18 g/d, P=0.004) had stabilized, mice were randomly assigned to receive the combination of the β1β2 blocker propranolol (2 mg/kg/d for 14 days) and β3 blocker SR59230A (1 mg/kg/d for 14 days) or vehicle delivered by osmotic minipumps. The β blockade efficiently attenuated the hypothalamic BDNF-induced gene program in white adipose tissue (FIG. 11E).

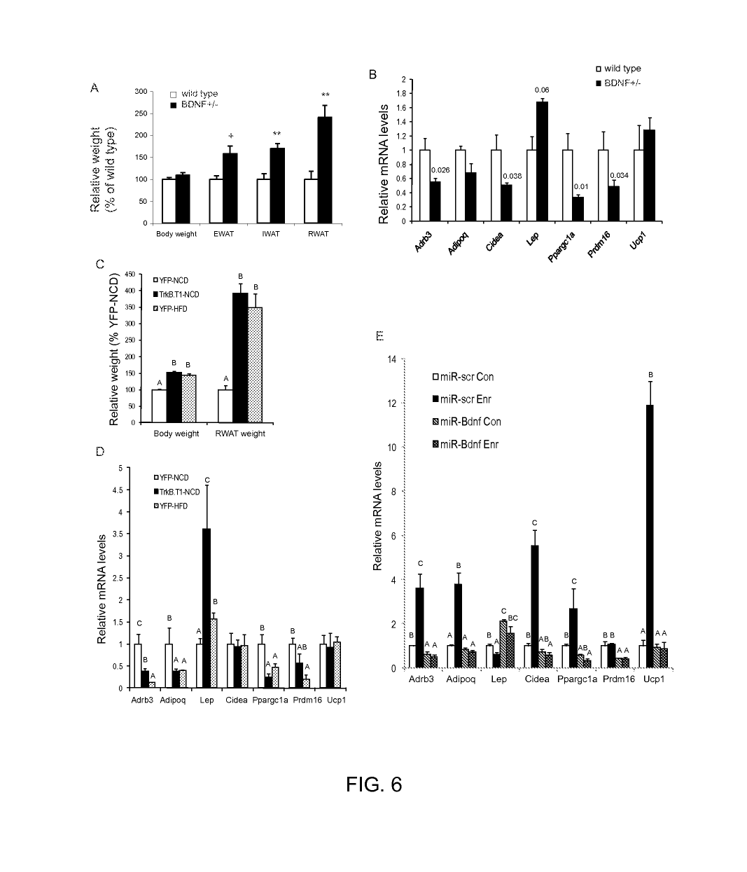

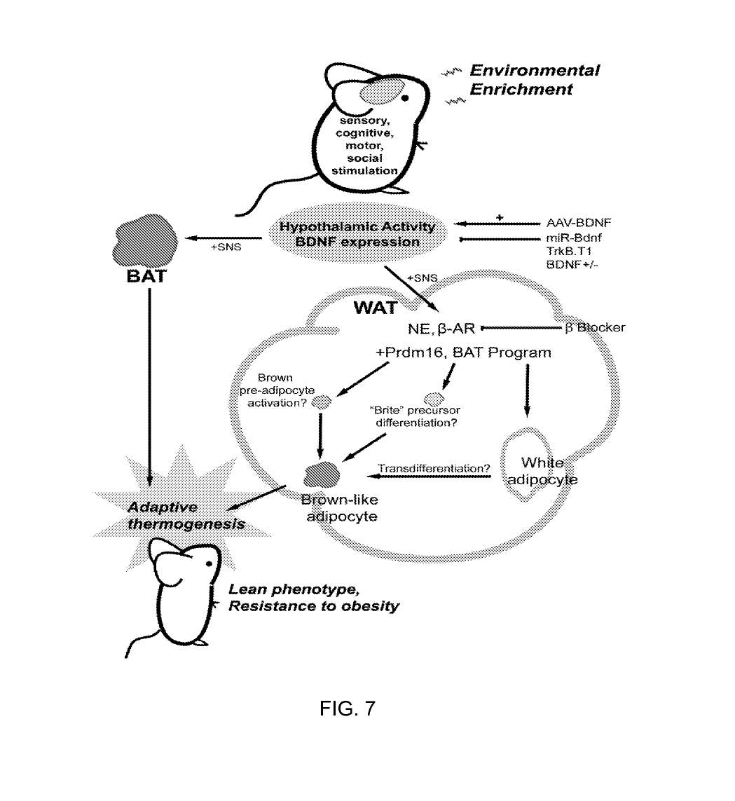

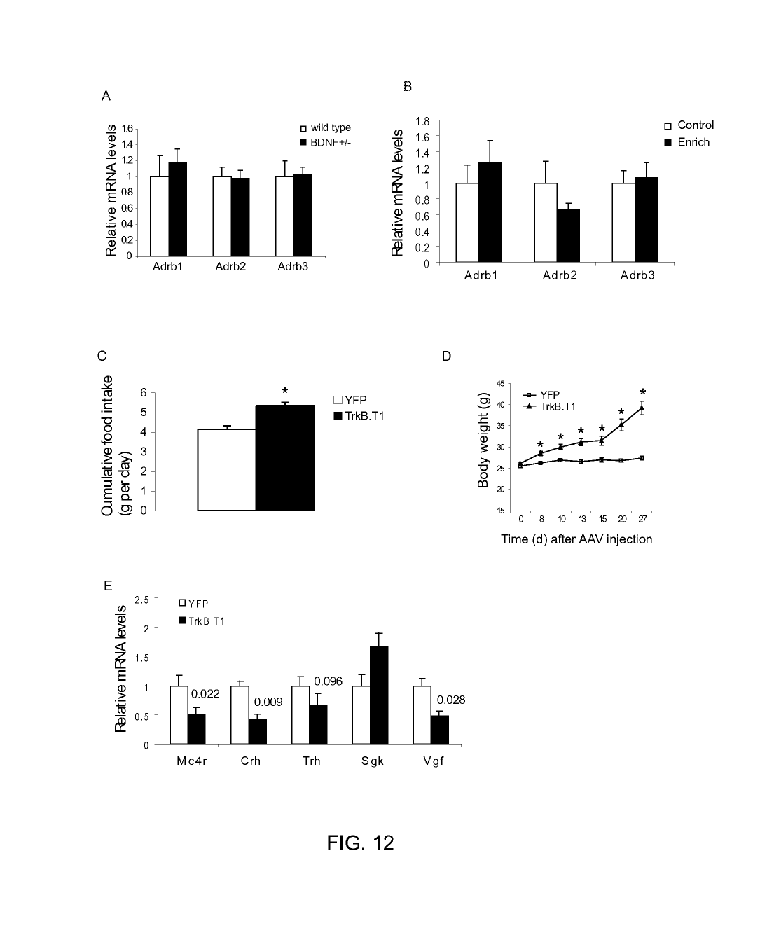

To further define the role of BDNF we evaluated several strategies including the use of BDNF heterozygous mice (BDNF+/−) that develop adult-onset obesity associated with hypothalamic BDNF protein levels approximately 40% lower than wild type (Lyons et al., 1999). We observed a substantial increase in the fat pad mass of BDNF+/− mice before a significant body weight difference occurred (FIG. 6A). The molecular features of retroperitoneal white adipose tissue in BDNF+/− mice were a complete reversal of that found with EE or BDNF-overexpressing mice, namely a suppression of β-ARs and brown adipose tissue gene program (FIG. 6B). However, altered expression of β-ARs was not observed in muscles of either BDNF+/− mice (FIG. 12A) or EE mice (FIG. 12B) suggesting a selective modulation of fat β-AR signaling. We then used a dominant negative truncated form of the high affinity BDNF receptor (TrkB.T1) to specifically inhibit BDNF signaling in the hypothalamus of adult mice. Mice receiving rAAV-TrkB.T1 consumed more food (FIG. 12C) and gained more weight than AAV-YFP controls (FIG. 12D). Hypothalamic expression of TrkB.T1 reversed the gene expression changes associated with BDNF overexpression in the hypothalamus (FIG. 12E, FIG. 11D), suggesting efficient inhibition of BDNF signaling. Similar to DIO mice, retroperitoneal white adipose tissue was enlarged in TrkB.T1 mice associated with the obesity. The molecular signature of retroperitoneal white adipose tissue was similar to BDNF+/− mice (FIG. 6B). In order to examine whether the observed changes in retroperitoneal white adipose tissue was simply a result of obesity, we compared the retroperitoneal white adipose tissue molecular features between the DIO model receiving YFP control virus and TrkB.T1-induced obesity model. Although both models were identically obese (FIG. 6C), TrkB.T1 led to a further increase of Lep expression above that of the DIO model (FIG. 6D). Thus both global and hypothalamic-specific inhibition of BDNF led to a complete reversal of the EE-associated molecular features in white adipose tissue indicating BDNF's involvement in white adipose tissue gene program regulation. Furthermore we investigated whether BDNF mediated the EE-associated molecular “browning” of white adipose tissue by using microRNA to block EE-induced BDNF upregulation in hypothalamus (Cao et al., 2009). We generated AAV vectors expressing a microRNA targeting Bdnf (miR-Bdnf) and a microRNA targeting a scrambled sequence (miR-scr.) (Cao et al., 2010). We injected rAAV vectors of miR-Bdnf or miR-scr. into the hypothalamus and then assigned the mice to standard or EE housing for 4 weeks. MicroRNA knockdown of BDNF led to accelerated weight gain by approximately 2 fold. In mice receiving miR-scr, the EE-induced molecular signatures of retroperitoneal white adipose tissue was maintained (FIG. 6E). In contrast miR-Bdnf efficiently inhibited EE-induced hypothalamic BDNF upregulation (Cao et al., 2010) and completely blocked the molecular changes in white adipose tissue associated with EE (FIG. 6E).

### Prophetic Example 8

A subject is administered via stereotactic injection a vector expressing BDNF, or a BDNF analog, directly into the hypothalamus. The vector could be any viral or non-viral delivery method for entry of nucleic acid into target cells within the brain. Following the injection of the vector, the cell's transcriptional machinery would drive production of the BDNF mRNA, and translation to mature BDNF protein.

Transduced cells within the hypothalamus release the BDNF protein, which via its actions both locally, and via axons to efferent regions would increase trkB signaling resulting in activation of the hypothalamic-sympathoneural-adipocyte (HSA) axis as described. This HSA axis activation would lead to the increased phenotypic conversion of white adipocytes towards a brown adipocyte fate.

This increased “browning” can be observed via both an increase in the basal metabolism, increased resting oxygen consumption using metabolic chambers, or alternatively using fluorodeoxyglucose (FDG) PET, wherein increased metabolism is observed in white fat depots. This increased energy consumption translated to loss of fat, and reduced body weight.

### Prophetic Example 9

A subject would be administered a systemic dose of a BDNF mimetic, for example, LM22A-4. This can be administered perorally, or via subcutaneous, intramuscular, intravenous or intranasal routes. A dose of 0.1 to 1 g can be given, with the preferred dose in the 10-500 mg range. Following administration of this mimetic, activation of the HSA axis is expected to occur, again resulting in the metabolic changes, increased in white fat browning and measurable via basal metabolic rate monitoring and FDG PET. As a result, obese subjects are expected to lose excessive body weight.

### Prophetic Example 10

A subject will be administered a beta-3 agonist, CL-316,243, orally, or via intravenous, subcutaneous, intravenous, or intranasal routes. The drug may be administered at any dose from 0.1 to 1 g, with the preferred dose in the 10-1000 mg range. As a result of administration of the agonist, the molecular browning of white adipocytes is expected to be induced, with increased metabolic rate of the white adipocyte tissue depots. This will be reflected in the subject losing body weight for a given calorie intake, and increased basal metabolism and brown fat as measured by PET.

We claim:

1. A method for increasing the amount of brown adipose tissue in a subject having a low amount of brown adipose tissue, the method comprising administering a TrkB receptor agonist or a nucleotide sequence encoding the TrkB receptor agonist to a subject having a low amount of brown adipose tissue to white adipose tissue, wherein the TrkB receptor agonist or expression of the TrkB receptor agonist increases the amount of brown adipose tissue in the subject.

2. The method of claim 1, wherein the TrkB receptor agonist is administered as a peptide.

3. The method of claim 2, wherein the peptide is selected from the group consisting of BDNF, Neurotrophin-4, and Neurotrophin-3.

4. The method of claim 3, wherein the peptide is BDNF.

5. The method of claim 1, wherein the nucleotide sequence is administered encoding BDNF, Neurotrophin-4, Neurotrophin-3, or a combination thereof.

6. The method of claim 5, wherein the nucleotide sequence encodes BDNF.

7. The method of claim 1, wherein the TrkB receptor agonist or the nucleotide sequence encoding the TrkB receptor agonist is administered systemically, centrally, directly into a brain, or a combination thereof in an amount sufficient to activate the TrkB receptor.

8. The method of claim 1, wherein the TrkB receptor agonist or expression of the TrkB receptor agonist modulates sympathetic nervous system activity, thereby increasing the amount of brown adipose tissue in the subject.

9. The method of claim 1, wherein the TrkB receptor agonist or expression of the TrkB receptor agonist modulates a hypothalamic BDNF signaling pathway in the subject.

10. The method of claim 1, wherein the TrkB receptor agonist is selected from the group consisting of 7,8-dihydroxyflavone, N-acetylserotonin, Amitriptyline, 4′-Dimethylamino-7,8-dihydroxyflavone, LM22A-1, LM22A-2, LM22A-3, and LM22A-4.

11. The method of claim 1, wherein the subject shows enhanced weight loss.

12. The method of claim 1, wherein the subject is suffering from diet-induced obesity.

13. A method for increasing the amount of brown adipose tissue in a subject, the method comprising administering a beta-3 adrenergic receptor agonist to a subject having a low amount of brown adipose tissue, wherein the beta-3 adrenergic receptor agonist increases the amount of brown adipose tissue in the subject.

14. The method of claim 13, wherein the beta-3 adrenergic receptor agonist is selected from the group consisting of Octopamine, Amibegron, Solabegron, Nebivolol, L796568, CL-316243, LY-368842, and Ro40-2148.

15. The method of claim 13, wherein the beta-3 adrenergic receptor agonist is administered orally or by injection in an amount sufficient to activate the beta-3 adrenergic receptor.

16. The method of claim 13, wherein the beta-3 adrenergic receptor agonist is administered in an amount sufficient to reduce the amount of white adipose tissue.

17. The method of claim 13, wherein administering the beta-3 adrenergic receptor agonist modulates β-adrenergic signaling, thereby causing a browning of white adipose tissue.

18. The method of claim 13, wherein the subject is suffering from diet-induced obesity.

19. A method of increasing the amount of brown adipose tissue in a subject, the method comprising administering an agonist or a nucleotide sequence that encodes an agonist that modulates a hypothalamic-adipocyte axis in a subject having a low amount of brown adipose tissue, wherein modulation of the hypothalamic-adipocyte axis increases the amount of brown adipose tissue in the subject.

20. The method of claim 19, wherein the agonist is a TrkB receptor agonist or a beta-3 adrenergic receptor agonist.

21. The method of claim 20, wherein the TrkB receptor agonist is a peptide selected from the group consisting of BDNF, Neurotrophin-4, and Neurotropin-3.

22. The method of claim 21, wherein the peptide if BDNF.

23. The method of claim 20, wherein the TrkB receptor agonist is selected from the group consisting of 7,8-dihydroxyflavone, N-acetylserotonin, Amitriptyline, 4′-Dimethylamino-7,8-dihydroxyflavone, LM22A-1, LM22A-2, LM22A-3, and LM22A-4.

24. The method of claim 19, wherein the nucleotide sequence encodes BDNF, Neurotrophin-4, Neurotrophin-3, or a combination thereof.

25. The method of claim 24, wherein the nucleotide sequence encodes BDNF.

26. The method of claim 20, wherein the beta-3 adrenergic receptor agonist is selected from the group consisting of Octopamine, Amibegron, Solabegron, Nebivolol, L796568, CL-316243, LY-368842, and Ro40-2148.

27. The method of claim 19, wherein the agonist or expression of the agonist decreases an amount of white adipose tissue in the subject.

28. The method of claim 19, wherein the subject is suffering from diet-induced obesity.

29. The method of claim 19, wherein the agonist or expression of the agonist increases or induces expression of BDNF, thereby increasing the amount of brown adipose in the subject.

30. A method of preventing weight gain or effecting weight loss in a mammal, the method comprising providing an enriched environment to a mammal, wherein the presence of the enriched environment increases the amount of brown adipose tissue in the mammal.
